SEQUENCE LISTING (2 sequences)

SEQ ID NO: 1 - DNA, 11 bp, synthetic construct
  source 1..11: mol_type = other DNA
gtttaggctgt

SEQ ID NO: 2 - DNA, 20 bp, synthetic construct
  source 1..20: mol_type = other DNA
gatttaaattaaagaattac

(19) United States
(12) Patent Application Publication
(10) Pub. No.: US 2026/0071208 A1
(43) Pub. Date: Mar. 12, 2026

(54) METHODS OF PHAGE GENE MANIPULATION AND PROFILING

(71) Applicant: Wisconsin Alumni Research Foundation, Madison, WI (US)

(72) Inventors: Srivatsan Raman, Middleton, WI (US); Jackie Chen, Chicago, IL (US)

(73) Assignee: Wisconsin Alumni Research Foundation, Madison, WI (US)

(21) Appl. No.: 19/326,991

(22) Filed: Sep. 12, 2025

Related U.S. Application Data
(60) Provisional application No. 63/693,829, filed on Sep. 12, 2024.

(51) Int. Cl. C12N 15/10; C12N 9/22; C12N 15/11; C12N 15/90

(52) U.S. Cl. CPC C12N 15/1082; C12N 9/222; C12N 15/1086; C12N 15/111; C12N 15/902; C12N 2310/20

(57) ABSTRACT

Described herein is the Phage high-throughput approach for gene essentiality Mapping and Profiling (PhageMaP) platform to generate pooled, barcoded phage knockouts for high throughput screening of conditional phage gene essentiality. A barcoded donor plasmid library is prepared wherein each member includes a single guide RNA (gRNA) spacer sequence and 5′ and 3′ homology arm sequences. A host cell engineered to express a Cas nuclease and a recombinase is then transformed with the donor plasmid library followed by infection with a population of target phage to provide a barcoded target phage variant library produced by double stranded cleavage of the target phage genomes by the Cas nuclease-gRNA and subsequent recombinase-mediated homologous recombination with the donor plasmid. Insertion of the barcodes at the genomic loci disrupts the function of the genomic loci and provides the barcoded target phage variant library which may then be isolated.

### CROSS-REFERENCE TO RELATED APPLICATIONS

This application claims priority to U.S. Provisional Application 63/693,829 filed on Sep. 12, 2024, which is incorporated herein by reference in its entirety.

### STATEMENT REGARDING FEDERALLY SPONSORED RESEARCH & DEVELOPMENT

This invention was made with government support under AI156785 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Aug. 7, 2025, is named “SEQ_LIST--107668335-P240244US02.xml” and is 2,809 bytes in size. The Sequence Listing does not go beyond the disclosure in the application as filed.

### FIELD OF THE DISCLOSURE

The present disclosure is related to high throughput methods for screening of genes in phage genomes, particularly the screening of essential genes for application in phage engineering.

### BACKGROUND

Bacteriophages, or phages, are highly diverse and abundant bacterial viruses that can be used in a broad range of applications such as precision microbiome editing, biofilm degradation, gene delivery, and therapy for multidrug-resistant infections. Despite over a century of phage research, our understanding of how phages infect bacterial populations remains limited. The core problem is not knowing which phage genes are essential, non-essential, or amenable to editing. Furthermore, research in recent years has revealed that bacteria possess a wide array of anti-phage defense systems that protect the host cell against infection. How these systems interact and interfere with the phage genome remains poorly understood. Together, these gaps in knowledge hamper our ability to engineer phage for industrial and biomedical use.

Recent advances in high-throughput sequencing have accelerated the discovery of new phages. Gene knockout screens are typically performed by introducing transposases or CRISPR-Cas9 to disrupt genes. However, in the case of phages, which lack cellular machinery, creating gene knockout libraries has been challenging. Further, obligate lytic phages are harder to manipulate because they do not integrate into the host genome, rendering tools for bacterial genome engineering, such as recombineering, ineffective.

What is needed are high throughput methods for screening of genes in phage genomes. A scalable molecular tool would be highly beneficial in assessing gene essentiality during infection in the presence of antiphage defense and for the functional annotating of uncharacterized genes, both of which can inform more effective phage engineering.

### BRIEF SUMMARY

In an aspect, a method of preparing a barcoded target phage variant library, comprises
providing a donor plasmid library, wherein each member of the donor plasmid library is a bacterial vector comprising a single guide RNA (gRNA) spacer sequence, a 3′ homology arm sequence, and 5′ homology arm sequence, wherein the gRNA spacer and homology arms define a Cas nuclease cleavage site at a genomic locus in the target phage;
inserting a barcode insert into each member of the donor plasmid library between the 3′ and 5′ homology arms to provide a barcoded donor plasmid library, wherein the barcode insert comprises a barcode flanked by barcode primer binding sites;
transforming a host cell with the barcoded donor plasmid library to provide a transformed host cell, wherein the host cell is engineered to express a Cas nuclease and a recombinase,
infecting the transformed host cell with a population of target phage to provide the barcoded target phage variant library, wherein double stranded cleavage of the target phage genomes by the Cas nuclease-gRNA and subsequent recombinase-mediated homologous recombination with the donor plasmid inserts the barcodes into the genomic loci in the target phage, wherein insertion of the barcodes at the genomic loci disrupts the function of the genomic loci; and
isolating the barcoded target phage variant library.

In another aspect, a method of multiplexed, targeted phage genome manipulation and profiling comprises providing a barcoded target phage variant library, wherein each member of the barcoded target phage variant library comprises a barcode inserted at a genomic locus in the target phage, wherein insertion of the barcode at the locus disrupts the function of the locus, challenging a panel of susceptible bacterial hosts or a single susceptible bacterial host under a plurality of conditions with the barcoded target phage variant library, and quantifying the pre-challenge and post-challenge abundances of one or more phage variants from the barcoded target phage variant library.

### BRIEF DESCRIPTION OF THE DRAWINGS

FIGS. 1A-F show generating phage knockout libraries using PhageMaP. (1A) Scheme for generating barcoded phage knockout libraries. Oligos containing target-specific 95 nt homology arms and sgRNAs are cloned into a high-copy number vector. 16N barcodes are subsequently added to generate a barcoded donor plasmid library. This library is infected by WT T7 and Cas9-RecA-mediated homologous recombination (HR) inserts barcodes into designated loci. (1B) Screening of the PhageMaP libraries on diverse hosts. The pre- and post-selection abundances of phage variants are quantified with deep sequencing. Each variant is then scored to create context-specific gene essentiality profiles. (1C) Validation of barcode insertions into four T7 genomic loci. Cas9-RecA-mediated HR was carried out using 1 of 4 sgRNAs or a mix of the 4 sgRNAs in pooled format. The four genomic loci were sequenced, and the percentage of recombined phages was determined by finding the ratio of insertions to total reads. (1D-F) Distribution and mapping of the T7, Bas63, and T4 PhageMaP libraries.

FIGS. 2A-G show screening the T7 PhageMaP library on a panel of common laboratory E. coli strains. (2A) Heatmap of fitness scores after Z-normalization for eleven hosts. A 10th percentile floor and 90th percentile ceiling were imposed for the scaling. Lines through each cell represent the standard error (SE), where a full line represents SE≥1. Clustering was performed using the Euclidean metric and Ward linkage method. Metadata describe the general classification of each gene. Black boxes indicate no data were obtained for the gene in the corresponding condition. (2B) Plot mapping the genomic coordinates and scores for intergenic perturbations. The points (intergenic variants) and blocks (genes) correspond to (2A). (2C) Example from (2A) of generally nonessential genes that become more essential. The presence of intact Type I restriction-modification systems was determined by using PADLOC. (2D-G) Example from (2A) of (2D) generally essential genes that become more nonessential, (2E) moderately essential gene becoming more essential, (2F) resolution of overlapping genetic elements, and (2G) unclear conditional essentiality.

FIGS. 3A-C show screening the Bas63 PhageMaP library on a panel of common laboratory E. coli strains. (3A) Heatmap of the Bas63 fitness scores after Z-normalization within each of the 4 conditions. To better emphasize differences outside the two extremes of the data, a 10^{th} percentile floor and a 90^{th} percentile ceiling were imposed for the scaling. The lines through each cell represent the standard error (SE) where a full line represents SE≥1. Clustering was performed using the Euclidean metric and Ward linkage method. Metadata describes general classification of each gene. Black boxes indicate no data was obtained for the gene in the corresponding condition. A black dot at the top right corner of a cell indicates only one replicate was obtained. Asterisks at the end of gene names indicate genes which were manually annotated after BLASTp search. (3B) Genome map colored by the median fitness score for each gene from the tested conditions. Scaling is the same as (3A). (3C) Scatterplots showing the fitness scores and SEs for each fitness measurement for the four hosts. Dotted lines represent the chosen threshold for “nonessential” and “essential” genes.

FIGS. 4A-C show screening the T4 PhageMaP library on a panel of common laboratory E. coli strains. (4A) Histogram showing distribution of genome sizes across all “complete” and “high-quality” sequenced genomes found in the PhageScope database (>370 K genomes). Genome sizes of the phages used in this study are indicated by the dotted lines. The percentile among all genome sizes is indicated. (4B) Heatmaps of the Z-normalized T4 fitness scores across six hosts, organized by putative function. A 10 th percentile floor and a 90 th percentile ceiling were imposed for the scaling. Lines through each cell represent the standard error (SE), where a full line represents SE≥1. Clustering was performed using the Euclidean metric and Ward linkage method. Black boxes indicate no data were obtained for the gene in the corresponding condition. A black dot at the top right corner of a cell indicates only one replicate was obtained. (4C) T4 genome map coded by the median fitness score for each gene across tested hosts, the same as (B).

FIGS. 5A-S show identification of anti-phage defense counters and triggers in T7 and Bas63 phages. (5A) Identification of counters and triggers. (5B) Trigger for the PARIS defense system from the T7 dataset. Difference between raw fitness scores is shown. (5C) RADAR2 trigger from the T7 dataset. (5D) Inosine quantification in empty vector or RADAR2 cells after repression or induction of T7gp5.7. Data are represented as mean±SD of three independent replicates. p values (Welch's t test) are annotated. (5E) Sir2-HerA counters from the Bas63 dataset. (5F) Efficiency of plating (EOP) measurements for Bas63 knockouts with the Sir2-HerA host. Data are represented as mean±SD of three independent replicates. Hypothesis testing was performed between each knockout and WT. P values (Welch's t test) and means are annotated. Complementation host contained the defense system and a plasmid expressing deleted gene. (5G) Model for Sir2-HerA. (5H) Ec86 retron counters from the Bas63 dataset. (5I) EOP measurements for Bas63 knockouts with the Ec86 retron host. Calculated same as (F). (5J) Model for Ec86 retron. (5K) Ec67 retron counter from the Bas63 dataset. (5L) EOP measurements for a Bas63 knockout with the Ec67 retron host. Calculated same as (5F). (M) Model for Ec67 retron. (5N) QatABCD counters from the Bas63 dataset. (5O) EOP measurements for Bas63 knockouts with the gatABCD host. Calculated same as (5F). (5P) Model for gatABCD. (5Q) Ppl counter from the Bas63 dataset. (5R) EOP measurements for a Bas63 knockout with the ppl host. Calculated same as (5F). (5S) Model for ppl. Volcano plots show the difference between Z-normalized fitness scores in defense and no-defense conditions unless specified otherwise.

FIGS. 6A-C show the successful transfer of a counter from Bas63 phage to improve activity of T7 phage during ppl anti-phage defense. (6A) Plaquing of T7 phage on no-defense (empty vector), ppl, ppl+GFP, and ppl+Bas63gp130 hosts and of T7Δ4.3::gp130 on no-defense and ppl hosts. (6B) EOP of T7 phage and T7Δ4.3::gp130 on no-defense, ppl, and ppl+Bas63gp130 hosts. For each phage, the no-defense is used as the reference for EOP calculations. Data are represented as mean±SD of three independent replicates. p values are calculated using Welch's t test. (6C) Diameter of lysis zones with T7 phage and T7Δ4.3::gp130 on no-defense, ppl, ppl+sfGFP, and ppl+Bas63gp130 hosts. Data are represented as mean±SD of three independent replicates. p values are calculated using Welch's t test.

FIG. 7 shows the improved recombination efficiency and the workflow to obtain measurements. Cells containing donor plasmids that would replace a 100 bp fragment of g1.1 in T7 with a 100 bp primer binding sequence-flanked barcode were co-transformed with a Cas9-sfGFP or Cas9-RecA plasmid. Cells were either uninduced or induced with 1% anhydrotetracycline. The cultures were infected by WT T7 phage and the resulting progeny phages were used as template for PCR amplification of the target locus for sequencing. Recombined percentages were calculated as percent of total reads that contain a consensus region found in the barcode insertion. Bars represent the average (n=3 biological replicates) recombined percentages. Error bars denote standard deviation. LOD is the limit of detection which is 1 read.

FIGS. 8A-D show the analysis of PhageMaP library quality for T7, Bas63, and T4 phages. (8A) Rank-order curves for the library members at the donor plasmid and phage library stages. (7B) Total barcode counts at the donor plasmid and phage library stages. (7C) sgRNA representation for each gene at the donor plasmid and phage library stages. (7D) Estimation of recombination rate of phage libraries with digital droplet PCR (ddPCR).

The above-described and other features will be appreciated and understood by those skilled in the art from the following detailed description, drawings, and appended claims.

### DETAILED DESCRIPTION

As described herein the Phage high-throughput approach for gene essentiality Mapping and Profiling (PhageMaP) is a platform to generate pooled, barcoded phage knockouts for high throughput screening of conditional essentiality.

PhageMaP is systematic, high-throughput, generalizable, and versatile. For example, the use of Cas9-recombinase-mediated homologous recombination (HR) for mutagenesis enables systematic interrogation of user-defined genetic elements by simply designing the appropriate single guide RNAs (sgRNAs) and donor template. PhageMaP's high-throughput nature is derived from the pooling of sgRNA and barcoded donor sequences targeting all genes across the phage genome which allows for the creation of barcoded genome-wide knockouts in a single culture. PhageMaP can be generalized to other phages, provided Cas9 and recombinases can function within a permissive host—a condition met by many common bacterial hosts. Finally, PhageMaP is versatile because the same phage library, generated once, can be applied to a myriad of conditions and sequenced directly from the lysate in a manner reminiscent of transposon insertion libraries used for high-throughput phenotyping and identification of conditionally essential genes in bacteria and yeast.

More specifically, described herein is a method referred to as Phage high-throughput approach for gene essentiality Mapping and Profiling (PhageMaP), a method for generating barcoded knockout libraries for user-defined loci within a phage genome through homologous recombination (HR)-guided insertional mutagenesis. PhageMaP creates barcoded deletion libraries for each phage during its replication cycle within a permissive host. During phage replication, Cas9 induces a double stranded break at the target site on the phage genome, which is subsequently repaired by the donor cassette containing a barcode. The resulting progeny phages carry barcoded deletion mutants of the target gene. PhageMaP is generalizable to other phages, both lytic and lysogenic, as the only requirements are the ability of Cas9 to function and the capacity to undergo homologous recombination in a permissive host, a condition met by diverse bacterial hosts.

In an aspect, described herein is a phage-based method for high throughput screening of conditional essentiality where DNA barcodes are systematically inserted into defined loci of a phage genome using in vivo Cas9-RecA-mediated HR—effectively creating knockouts whose abundances can be quantified via highly-multiplexed deep sequencing of the inserted barcodes. High-fidelity HR and Cas9-targeting enable systematic perturbations at desired positions. Advances in oligo synthesis technology allow for the comprehensive tiling of a phage genome with single guide RNAs (sgRNA) and the construction of the appropriate donor DNA. Barcoding the phage variants for direct phage-based readout permits facile testing against numerous hosts with relatively small sequencing volumes (˜2M reads per sample). In these screens, a phage carrying a barcode that inactivates an essential gene for replication on a host will deplete, thus reducing the barcode count for that variant. In contrast, a phage with a barcode within a nonessential region should persist, thus higher counts of that barcode are expected. By quantifying the change in barcode abundance post-selection, the fitness for each gene in the tested condition can be calculated.

As described herein, PhageMaP was used to generate pooled genome-wide knockout libraries for model (T7 and T4) and non-model (Bas63) E. coli phages. Phage libraries were challenged with two separate host panels: an 11-member panel consisting of clinically relevant and common laboratory E. coli strains and a 33-member panel of a strain of E. coli harboring anti-phage defense systems. PhageMaP recapitulated the patterns of gene essentiality established from decades of research, such as the indispensability of core structural and replication proteins and the conditional essentiality of phage defense inactivators. Potential novel phage defense interactors were also identified. PhageMaP can be used to identify and characterize molecular players of complex phage-host interactions across diverse experimental conditions such as different bacterial strains and anti-phage defense systems.
an aspect, a method of preparing a barcoded target phage variant library, comprises
providing a donor plasmid library, wherein each member of the donor plasmid library is a bacterial vector comprising a guide RNA (gRNA), 3′ homology arm and 5′ homology arm, wherein the gRNA spacer and homology arms define a Cas nuclease cleavage site at a genomic locus in the target phage,
inserting a barcode insert into each member of the donor plasmid library between the 3′ and 5′ homology arms to provide a barcoded donor plasmid library, wherein the barcode insert comprises a barcode flanked by barcode primer binding sites, and
transforming a host cell with the barcoded donor plasmid library to provide a transformed host cell, wherein the host cell is engineered to express a Cas nuclease and a recombinase,
infecting the transformed host cell with a population of target phage to provide the barcoded target phage variant library, wherein double stranded cleavage of the target phage genomes by the Cas nuclease-gRNA and subsequent recombinase-mediated homologous recombination with the donor plasmid inserts the barcodes into the genomic loci in the target phage, wherein insertion of the barcodes at the genomic loci disrupts the function of the genomic loci, and
isolating the barcoded target phage variant library.

Exemplary target phage includes lytic page and lysogenic phage. In general, lytic phages are very species-specific with regard to their hosts and typically infect a single, bacterial species, or even specific strains within a species. Exemplary lytic phages that infect E. coli include phiX174, T1, T2, T3, T4, T5, T6 and T7 bacteriophages. Sb-1 and Pyo phages infect S. aureus including methicillin-resistant Staphylococcus aureus (MRSA) strains. Dp-1 and Cp-1 are lytic bacteriophages for S. pneumoniae. Phage HEf13 infects Enterococcus faecalis. CP26F and CP390 are lytic bacteriophage for Clostridium species. LL-H is a bacteriophage specific for Lactobacillus species. PM16 is specific for Proteus species. PhiKZ, PaPI and PADP4 infect Pseudomonas species. Phage SPP1 is specific for Bacillus species. Phages phiChi13, S16, and E1 are specific for Salmonella. Phages AP205, Fri1, and PD6A3 are specific for Acinetobacter species. Phages phiYeO3-12 and YpsP-PST are specific for Yersina species. Other phages such as D29 have been identified that infect Mycobacterium. CrAssphage and crass-like phages that infect Bacteroides.

Exemplary lysogenic phages that infect E. coli include lambda, P1, P2, Mu, and N15. Phage P22 and phi80 can infect Salmonella. Phage Ms6 and L5 are specific for Mycobacterium. Phages phiBT1, SV2, and phiC31 are specific for Streptomyces. Phage phi11 is specific for Staphylococcus. Phages YA3, phiPan70, and D3112 infects Pseudomonas. Phage SPbeta infects Bacillus. Phage ABMM1 infects Acinetobacter. phiCD6356, phiHN10, and phiCD6365 infect Clostridium species.

In an aspect, the target phage is a phage with a sequenced genome.

In the method, each member of the donor plasmid library is a bacterial vector comprising a guide RNA (gRNA) spacer sequence, a 3′ homology arm sequence and a 5′ homology arm sequence.

As used herein, the term guide RNA means the sequence of RNA that directs Cas9-mediated cleavage of target DNA. Short guide RNA (gRNA) sequences can complex with Cas9 nuclease to form a ribonucleoprotein (RNP) capable of producing double-strand breaks (DSBs) at a targeted location in the genome. Guide RNA, or gRNA, can be in the form of a crRNA/tracrRNA two guide system, or a single guide RNA (sgRNA). A guide RNA thus contains the sequences necessary for Cas9 binding and nuclease activity and a target sequence complementary to a target DNA of interest (protospacer sequence). In an aspect, the gRNA is an sgRNA.

In an aspect, the 3′ homology arm and 5′ homology arm have lengths of 50 to 150 nucleotides, such as 95 nucleotides. The length of the homology arms can readily be determined by one of ordinary skill in the art taking into account the size of the constructs, recombination efficiency, and potential toxicity for very long homology arms.

The gRNA spacer and homology arms define a Cas nuclease cleavage site at a locus in the target phage genome. As used herein, a Cas nuclease is the protein component of a CRISPR system, the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, gRNA interacts with a Cas nuclease (e.g., Cas9) and the ribonucleoprotein complex (RNP) directs the nuclease activity of the Cas nuclease to target DNA sequences complementary to those present in the gRNA. The gRNA base pairs with complementary sequences in target DNA. Cas nuclease activity then generates a double-stranded break in the target DNA.

A Cas nuclease is a polypeptide that functions as a nuclease when complexed to a guide RNA, e.g., an sgRNA or modified sgRNA. That is, a Cas nuclease is an RNA-mediated nuclease. The Cas9 (CRISPR-associated 9, also known as Csn1) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

For example, the Cas9 may include, a Cas9 from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the variant may preferably increase specificity; for example, SpyFi Cas9 (Aldevron, Fargo, N. Dak.). In an exemplary embodiment, the Cas9 includes Streptococcus pyogenes Cas9 (SpCas9).

Functional Cas9 mutants are described, for example, in US20170081650 and US20170152508, incorporated herein by reference for its disclosure of Cas9 mutants.

A barcode is then inserted into each member of the donor plasmid library between the 3′ and 5′ homology arms to provide a barcoded donor plasmid library. The barcode is inserted between the homology arms. Each barcode insert comprises a barcode flanked by barcode primer binding sites. Thus, each barcoded donor plasmid variant in the library comprises homology arms, gRNA, and a unique barcode.

As used herein, a barcode is a string of nucleotides which can be mapped to specific phage variants, deep sequenced from a mixed phage population, and used as a proxy readout for the abundance of a variant.

In an aspect, the barcodes have length of 16 to 24 nucleotides, although shorter and longer barcodes may be employed.

The barcodes can be inserted into protein coding and/or non-coding genomic loci.

In an aspect, the barcoded target phage variant library comprises members with barcode insertions in at least 50%, 55%, 60%, 65%, 70%, 75%, 80%, 85%, 90% or 95% of target phage genes. The method can further comprise mapping the barcodes of the members of the barcoded donor plasmid library to their loci in the target phage genome. In an aspect, mapping comprises sequencing the barcoded target phage variant library. This step may be omitted if barcode identities and their corresponding homology arms/sgRNAs are previously known.

Mapping can comprise direct sequencing of the barcoded donor plasmid library. In an aspect, short read sequencing such as Miseq 2×250 sequencing may be used. In another aspect, PacBio or Oxford nanopore long read sequencing can be used to assign the variant-barcode pairs.

Once the barcodes are inserted, the method includes transforming a host cell, e.g., E. coli, with the barcoded donor plasmid library to provide a transformed host cell, wherein the host cell is engineered to express a Cas nuclease and a recombinase. For example, prior to transforming the host cell with the donor plasmid library, the host cell may be transformed with a plasmid for expression of the Cas9 nuclease and the recombinase in the host cell. In an aspect, the host cell is deficient of endogenous recombinases.

In an aspect, the recombination competent host cell expresses a recombinase such as RecA. Recombinases direct homologous recombination and maintain genome integrity. RecA is an E. coli recombinase that binds single-stranded DNA at a DNA break and searches for homologous double-stranded DNA to provide a template for break repair. RecA requires ATP and Mg⁺² for its function. Recombinases homologous to RecA from the RAD51 superfamily such as Rad51in eukaryotes and Dmc1 in yeast can also be used. Recombinases from the Rad52 and Gp2.5 superfamilies can also be used. Variants of recombinases generated from mutagenesis or bioprospecting can also be implemented to improve recombination efficiency.

The transformed host cell is then infected with a population of target phage to provide the barcoded target phage variant library, wherein double stranded cleavage of the target phage genomes by the Cas nuclease-gRNA and subsequent recombinase-mediated homologous recombination with the donor plasmid inserts the barcodes into the genomic loci in the target phage, wherein insertion of the barcodes at the genomic loci disrupts the function of the loci. In an aspect, infection is performed at a multiplicity of infection (MOI) of 1 to 10. The MOI 5 is preferably selected to reduce library bias.

Once the barcoded target phage variant library is created, it can be isolated and sequenced. Preferably, each phage variant in the library is associated with 10 to 100 barcodes.

In another aspect, a method of multiplexed, targeted phage genome manipulation and profiling comprises providing a barcoded target phage variant library, wherein each member of the barcoded target phage variant library comprises a barcode inserted at a genomic locus in the target phage, wherein insertion of the barcode at the locus disrupts the function of the locus,
challenging a panel of susceptible bacterial hosts or a single susceptible bacterial host under a plurality of conditions with the barcoded target phage variant library, and
quantifying the pre-challenge and post-challenge abundances of one or more phage variants from the barcoded target phage variant library.

In an aspect, the choice of host and condition can be determined by the individual practicing the method. Any susceptible host or mix of bacterial hosts to the target phage can be used. Any changes to the growth conditions such as alterations to growth media components or environmental context can also be implemented.

In an aspect, the number of hosts and the number of conditions can be determined by the individual practicing the method.

In another aspect, the pre-challenge and post-challenge abundances can be determined across all hosts, or for each host individually.

In an aspect, the method can further include preparing a map of genomic locus essentiality by assigning a fitness score to each variant phage based on the pre-challenge and post-challenge abundances, wherein the fitness score is a measure of genomic locus essentiality. In an aspect, the statistical model from Enrich2 can be adapted. Barcode counts for each unique homology arm/sgRNA variant are summed and the normalized to the total read count. Normalized scores for each of those variants are used to calculate gene scores for that replicate using the maximum likelihood estimation model in Enrich2. These replicate gene scores are then passed through another round of maximum likelihood estimation to obtain the composite gene score. This composite gene score can then be further normalized such as by Z-score normalization for analysis across conditions.

In an aspect, the genomic loci in the variant phage are classified as essential, non-essential or conditionally essential. In an aspect, the essential and nonessential target genomic loci have fitness scores positioned on opposing ends of a numerical fitness scale. In another aspect, each conditionally essential target genomic locus has at least one fitness score that is positioned on an opposing end of a numerical fitness scale from the other fitness score(s) involving the same or related genomic locus.

Exemplary genomic loci can be in genes encoding enzymes involved in host takeover, enzymes involved in nucleotide metabolism, enzymes involved in DNA replication, structural proteins, and the like.

The invention is further illustrated by the following non-limiting examples.

### Examples

### Methods

Strains and culture conditions: Bacteriophage T7 was obtained from ATCC (ATCC BAA-1025-B2). Bacteriophage T4 was obtained from ATCC (ATCC 11303-B4). Bacteriophage Bas63 (Johann R Wettstein) was obtained from Alexander Harms (Biozentrum, University of Basel Biozentrum) and derived from the BASEL collection. E. coli DH10B (C3020) and E. coli BL21(DE3) (C2527) were purchased from NEB (C3020). E. coli BL21 (REL606) was obtained from Robert Landick (University of Wisconsin-Madison). E. coli S17-1 was obtained from Andrew Hryckowian (University of Wisconsin-Madison). E. coli BW25113 was obtained from Douglas Weibel (University of Wisconsin-Madison) and derived from the Keio collection. E. coli LE392 (bor::kanR) was obtained from Lanying Zeng (Texas A&M University). E. coli UTI33 and UTI46 were obtained from Rod Welch (University of Wisconsin-Madison) and derived from the UTI collection. The E. coli ECOR13 and ECOR4 strains were obtained from the Michigan State University STEC Center and derived from the ECOR collection. E. coli MG1655 is a laboratory strain. The phage defense strains in E. coli DH5a were a gift from Feng Zhang (Addgene plasmids #157880-157912) and the defense plasmids were subsequently transformed into MG1655. For long term storage, all bacterial strains were stored at −80° C. in 25% glycerol and 75% LB.

All bacterial strains were grown in LB media (1% tryptone, 0.5% yeast extract, 1% NaCl, and 1.5% agar for plates or 0.7% agar for top agar). If applicable, antibiotics kanamycin (Kan 50 μg/mL final concentration), chloramphenicol (Cam 25 μg/mL), and spectinomycin (Spec 100 μg/mL final concentration) were added for plasmid maintenance. Inducers anhydrotetracycline (1 μM final concentration) and L-arabinose (0.1% or 1% final concentration) were added if necessary for induction of pBAD promoter. D-glucose (0.2% or 0.5% final concentration) was added if necessary for repression of pBAD promoter. All incubations were performed at 37° C. and shaken at 200-250 rpm, if growing liquid cultures, unless specified otherwise.

The initial laboratory stock of bacteriophage T7 was propagated using E. coli BL21 after receipt from ATCC. New stocks were made by propagating the initial laboratory stock with E. coli DH10B. Bas63 was also propagated on DH10B. Propagation of both phages was performed using LB and culture condition described above. Phages were purified with 0.22 μm filters and stored at 4° C. in LB.

E. coli DH10B competent cells were prepared by inoculating 5 mL of overnight culture into 400 mL of SOB media (2% tryptone, 0.5% yeast extract, 10 mM NaCl, 2.5 mM KCl, 10 mM MgCl₂, 10 mM MgSO₄). Cells were grown at 37° C. shaking at 200 rpm until OD₆₀₀ ˜0.6 as determined by an Agilent Cary 60 UV-Vis Spectrometer and Ultrospec™ 10 Cell Density Meter (Amersham Biosciences). Cells were spun down at 5.5 k×g for 10 minutes at 4° C. Two washes with cold 10% glycerol were performed. Cells were spun down at 5.5 k×g for 10 minutes at 4° C. after each wash, and the supernatant was removed. The pellet was then resuspended with 1:100 starting volumes of cold 10% glycerol. Cells were aliquoted and stored at −80° C.

An estimate of 8×10⁸ CFU/mL at OD₆₀₀ 1 was used for quantifying cell numbers in a volume of culture in all experiments. Cell numbers were used to calculate the amount of phage to add for a target multiplicity of infection (MOI).

Double agar overlay plaque assays: Plaque assays were routinely performed to isolate phages and quantify titer of phage preparations. In general, 200-300 μL of stationary phase target host and a dilution of phage were added to 4 mL of 0.5-0.7% top agar (1% tryptone, 0.5% yeast extract, 1% NaCl, and 0.5-0.7% agar) with additional antibiotics, inducer, or glucose if applicable. The mix was then vortexed and plated on LB plates. The top agar was allowed to solidify (˜10 minutes) before incubation at 37° C. overnight. Plates containing 10 to 1000 plaques were typically counted. Titers were calculated by averaging at least 2 replicates.

General cloning procedures and base plasmid construction: All PCR reactions were performed using KAPA HIFI™ (Roche #KK2101) according to manufacturer instructions and with the following cycle settings unless specified otherwise: 95° C. 3 minutes, (98° C.→20 seconds→65° C. 15 seconds→72° C.×seconds)×25 cycles, 72° C.×seconds, and 4° C. hold, where X varies depending on amplicon length. An initial denaturation step of 5 min at 95° C. is used if the template is phage. A 2×KAPA HIFI™ master mix was made by adding dNTPs, HF Buffer, and KAPA HIFI™ DNA Polymerase and stored at −20° C. All PCR reactions use this master mix. DpnI (NEB #R0176L) digestion was performed if the template was derived from plasmid DNA by adding 1 μL DpnI and 2.3 uL 10×Cutsmart® Buffer directly into the PCR sample, incubating at 37° C. for 1 hour, and heat inactivating for 20 minutes at 80° C. DNA purification after PCR was performed either with the E.Z.N.A.® Cycle Pure Kit (Omega Bio-tek #D6492-01) or with gel extraction from 1% agarose gels using the E.Z.N.A.® Gel Extraction Kit (Omega Bio-tek D2500-01), both with a centrifugation protocol according to manufacturer instructions.

Golden Gate Assemblies were performed using the New England Biosciences (NEB) Golden Gate Assembly BsaI-HF® v2 (NEB #E1601L) or BsmBIv2 (NEB #E1601L) Kits. Reactions were prepared according to manufacturer instructions, but cycling was performed for 60 total cycles with 5-minute steps. Gibson Assemblies were performed using either in-house Gibson Master Mix (final concentration 100 mM Tris-HCl pH 7.5, 20 mM MgCl₂, 0.2 mM dNTP, 10 mM DTT, 5% PEG-8000, 1 mM NAD+, 4 U/mL T5 exonuclease, 4 U/uL Taq DNA ligase, 25 U/mL Phusion® polymerase) or NEB Gibson Assembly® Master Mix (NEB #E2611), following the Gibson Assembly@Protocol (NEB #E5510).

Following assembly of constructs, reactions were diluted 5-fold with dH₂O if performing plasmid transformation or dialyzed using MF-Millipore 0.025 μm MCE membranes (Millipore Sigma #VSWP02500) in dH₂O for 1 hour if performing donor library transformation or rebooting phage. For single plasmid transformations, 2 μL of the dilution was transformed into 25 μL DH10B electrocompetent cells. For library transformations, 5 μL of dialyzed reaction was transformed into 25 μL DH10B electrocompetent cells. For rebooting phage, half of each dialyzed reaction (˜10 μL) was transformed into 50 μL DH10B electrocompetent cells. Transformations were performed using a Bio-Rad MicroPulser™ (#165-2100). For single plasmid and library transformations, the Ec1 setting (1-mm cuvette, 1.8 kV) was used. For rebooting phage, the Ec3 setting (2-mm cuvette, 3 kV) was used. Recovery after electroporation was performed in a shaker at 200-250 rpm in a total volume of 1 mL SOC (2% tryptone, 0.5% yeast extract, 10 mM NaCl, 2.5 mM KCl, 10 mM MgCl₂, 10 mM MgSO₄, 20 mM glucose) for 30-60 min. For phage reboots, this step was omitted and 300 μL of transformants was added to 0.5% top agar and plated on LB plates. For library and single plasmid transformations, a diluted recovery was plated on LB plate with relevant antibiotics. Single colonies were picked and validated with PCR and/or sequencing prior to further outgrowth. Plasmid extraction was performed using the ZR Plasmid Miniprep™—Classic Kit (Zymo Research D4016) according to manufacturer instructions.

DNA quantification was performed using NanoDrop™ 2000 (Thermo Scientific) with 1.5 μl of DNA, except for Next Generation Sequencing DNA, which was quantified using a Qubi™ t 4 fluorometer (Thermo Scientific) with the Qubit™ 1×dsDNA HS Assay Kit (Thermo Scientific, #Q33231) following the manufacturer's documentation.

Following construction of non-library plasmids, plasmids were verified with either Sanger Sequencing (Functional Bioscience) or whole plasmid sequencing (Plasmidsaurus).

Construction of pUC19-Donor-BsmBI: To generate pUC19-Donor-BsmBI, the landing vector for oligos encoding donor and sgRNA information, two two-part Gibson Assembly® reactions were performed. In the first reaction, one fragment contains the pUC19 vector with the lacZa reporter gene removed, and the second fragment contains donor DNA and the sgRNA for one locus in T7. For the second Gibson reaction, one fragment is an amplicon that contains the sgRNA scaffold with the pUC19 vector generated from the first reaction. The second fragment contains an sfGFP gene flanked by new BsmBI cutsites and sequences homologous to the first two fragments. This version of the landing vector contains an ampicillin resistance cassette. This cassette was then swapped with a kanamycin cassette from a previously synthesized laboratory plasmid. Site-directed mutagenesis through Gibson Assembly® was done using this plasmid to remove the native BsaI recognition sites and generate the final pUC19-Donor-BsmBI plasmid. A variant of this plasmid (pUC19-Donor-BsaI) containing BsaI sites was also constructed using site-directed mutagenesis of pUC19-Donor-BsmBI and a 2-part Gibson Assembly®.

Construction of pCas9-RecA/sfGFP: A three-part Gibson Assembly® was used to generate tetracycline-inducible pCas9-RecA and pCas9-sfGFP. First the plasmid vector containing an SC101 origin, kanamycin resistance cassette, and the tet expression system was amplified with PCR from a previously generated laboratory plasmid. To obtain the Cas9 insert, a laboratory plasmid SC101-Cas9 was used as template for PCR. The recA insert was extracted from the pMP11 plasmid obtained from Brian Pfleger (University of Wisconsin-Madison). The sfGFP insert was extracted from the pUC19-Donor-Template plasmid.

pSM103 expression constructs: pSM103, which contains an arabinose-inducible pBAD promoter driving sfGFP, a kanamycin resistance cassette, and a pET31b origin of replication, was previously generated in the lab and used as the vector for insertion of phage genes for complementation and trigger studies. The vector was amplified with PCR. Phage genes were PCR amplified from 1 uL of phage stocks (˜10¹⁰ PFU/mL) and contain homology to the termini in the amplified pSM103 vector. The resulting fragments were assembled with Gibson Assembly®.

Donor plasmid library design and construction: Donor sequence design-T7 (V01146) and Bas63 (MZ501086) genome files were obtained from GenBank. To design sgRNAs for T7, DeepSpCas9 and CRISPRon gRNA efficiency prediction tools were used. The predicted efficiencies were concordant and CRISPRon only was used to design the T4 and Bas63 sgRNAs. For the T7 sgRNAs, a sliding window approach was implemented where the best available sgRNA (as determined by predicted efficiencies) was chosen from each 25 bp window. For the Bas63 and T4 libraries, the best 4 sgRNAs (as permitted by the gene) from the central 80% of each gene were chosen. For each sgRNA, the theoretical cut site (3 nt upstream of the PAM) was used as the starting position for the design of the 95 nt homology arms. The homology arms were designed such that the barcode will be inserted at the cut site position. The sgRNA, 5′ homology arm, 3′ homology arm, J23119 promoter, primer binding, and barcode landing sequences were combined with a custom Python script to create the donor plasmid insert sequences. These sequences were ordered as oligo pools (Twist Bioscience).

Construction of the barcode insert: The barcode insert contains a 16N sequence flanked by two constant sites containing stop codons in every reading frame and is used as primer binding sites to extract barcodes for sequencing. At the termini of the insert are BsmBI or BsaI cutsites for assembly into the donor plasmid vector. BC_oligo100, which contains the 16N barcodes, is first annealed and extended with either Twist_Bcoligo100_F (BsaI termini) or Twist_Bcoligo100_BsmBI_F (BsaI termini) by adding 1 μL of each oligo (100 μM) to 10 μL 2×KAPA HIFI Master Mix and 8 μL water. This mix was incubated at 95° C. for 30 seconds, 63° C. for 30 seconds, and then 72° C. for 30 seconds. The annealed oligos were then diluted 10-fold with the addition of 180 μL of water. 1 μL of the dilution was used as template with primer pairs (10 μM final concentration) Twist_Bcoligo100_F+Twist_Bcoligol00_R (BsaI termini) or Twist_Bcoligo100_BsmBI_F+Twist_Bcoligol00_BsmBI_R (BsmBI termini). PCR amplification was performed as follows: 95° C. 3 minutes, (98° C. 20 seconds→66° C. 15 seconds→72° C. 15 seconds)×20 cycles, 72° C. 15 seconds, and 4° C. hold. To mitigate PCR bubble formation, one additional round of PCR was performed after the addition of 1 uL of each primer. Each barcode insert was gel extracted and quantified as described above.

Insertion of the donor oligo sequences into pUC19 vector: To insert the T7 and T4 donor oligo sequences into the pUC19-Donor-BsmBI vector, a Golden Gate Assembly (BsmBI) was performed using 75 ng of pUC19-Donor-BsmBI and a 2-fold molar ratio of oligo insert. For Bas63 donor oligo sequences, pUC19-Donor-BsaI vector was used with Golden Gate Assembly (BsaI) Mix. The reactions were performed as described above. The reactions were dialyzed and transformed as described above. Recovery was performed in SOC for 30 minutes. 100 μL of the recovered transformants was plated on LB-Kan plates and the rest was used to inoculate 5 mL of LB-Kan media for overnight the growth. Plasmid DNA from the resulting outgrowth was extracted as described above. The resulting purified plasmids pUC19-T7 Library, pUC19-T4Library, and pUC19-Bas63Library are the landing vectors for the barcode insert.

Insertion of the barcode into donor vector: To insert the barcode insert (BsaI termini) into pUC19-T7Library or pUC19-T4Library, a Golden Gate Assembly (BsaI) was performed using 75 ng of pUC19-T7Library or pUC19-T4Library and a 2-fold molar ratio of barcode insert. For barcode insert (BsmBI termini), pUC19-Bas63Library vector was used with Golden Gate Assembly (BsmBI) Mix. The dialysis, transformation, and recovery outgrowth were handled the same as during the donor oligo sequence insertion. Serial dilutions of the recovered transformants were plated on LB-Kan plates and used to inoculate 5 mL of LB-Kan media for overnight growth. The dilution that gave approximately 10-fold coverage of the theoretical library size was used. Plasmid DNA from the resulting outgrowth was extracted as described above. The resulting plasmid libraries are pUC19-T7LibraryBC, pUC19-T4LibraryBC, and pUC19-Bas63LibraryBC.

Transformation of barcoded donor plasmid libraries into Cas9-RecA cells: E. coli DH10B Cas9-RecA competent cells were made the same way as described with E. coli DH10B with three differences. Cultures were scaled down to 10 mL, all growth steps occurred at 30° C., and the LB media used was supplemented with spectinomycin. 1 ng of barcoded donor library was transformed into 25 μL of Cas9-RecA competent cells. Transformants were recovered at 30° C. for 30 minutes. Serial dilutions of the recovered transformants were plated on LB-Kan-Spec plates and used to inoculate 5 mL of LB-Kan-Spec media for overnight growth. The dilution that gave approximately 10-fold coverage of the theoretical library size was used. Glycerol stocks for both libraries were made and stored at 80° C. Plasmid DNA from the resulting outgrowth was extracted as described above.

Sequencing donor plasmid libraries: To generate sequencing amplicons for both libraries, Ing of plasmid DNA was used as a template for a 10 μL PCR reaction with primer pairs DelLib{2,3,4}N_NGS_F+DelLib{2,3,4}N_NGS_R containing 2, 3, and 4 N offsets and the following PCR cycle settings: 95° C. 5 minutes, (98° C. 20 seconds→65° C. 15 seconds→72° C. 15 seconds)×15 cycles, 72° C. 1 minute, and 4° C. hold. Illumina® i7 and i5 adapter sequences containing unique indices and were added after performing a second 25 μL PCR reaction with the following cycle settings: 95° C. 3 minutes, (98° C. 20 seconds→65° C. 15 seconds→72° C. 15 seconds)×10 cycles, 72° C. 1 minute, and 4° C. hold. PCR products were purified with the E.Z.N.A.® Cycle Pure Kit and quantified using the Qubit™ 4 as described above. The purified products were pooled and sequenced on an Illumina® MiSeq with a 2×250 Miseq Reagent v2 kit.

Sequencing data processing and analysis: Read pairs were merged and filtered with Fastp, requiring a minimum of 90% of the read having scores above Q25. The adapter sequences were removed, and relevant donor sequences were extracted using Cutadapt. The resulting sequences were further analyzed using custom Python scripts. The result is the mapping of barcodes to their theoretical insertion site (coding or noncoding regions).

Generating PhageMaP phage libraries: Homologous recombination with donor libraries: For both T7, T4, and Bas63 PhageMaP libraries, 5 mL of LB-Kan-Spec was inoculated with 100 μL of pUC19-Bas63Library+pCas9-RecA or pUC19-T7Library+pCas9-RecA thawed glycerol stock. Both cultures were grown overnight at 30° C. The T7 donor library was added to 800 mL of LB-Kan-Spec, and the T4/Bas63 donor libraries were added to 500 mL of LB-Kan-Spec. Both were grown at 30° C. until the OD₆₀₀ reached 0.3-0.5. Anhydrotetracycline was added to each culture to a final concentration of 1 μM to induce Cas9 and RecA. Cultures were grown at 30° C. until the OD₆₀₀ reached ˜0.6. WT phage was added to their respective libraries at an MOI of 5. We reasoned that infection of the recombination host at a high multiplicity of infection (MOI 5) would enable phage variants with knockout of essential genes to persist in the population due to complementation from secondary co-infections. After infection of the donor host with wildtype phage, the genomes are inactivated by the encoded Cas9-sgRNA pair to create double stranded breaks used for HR and reduction of wildtype background. Inactivated phage genomes undergo RecA-mediated HR with the intracellular donor plasmids to restore genome integrity and to insert barcodes into target locus. After a 2-hour incubation at 37° C., lysates were transferred to centrifuge bottles and centrifuged at 16,000×g for 15 minutes at 4° C. to remove cell debris. The supernatant was then filtered using 0.45 m PES membrane filters.

Cesium Chloride (CsCl) density gradient: CsCl density gradients were used to concentrate and purify phage libraries. To remove free nucleic acids, lysates were first treated with DNase I (Roche #10104159001, 1 μg/mL final concentration) and RNase A (Fisher Scientific #NC972993, 10 μg/mL) after the addition of CaCl₂ (500 PM) and MgCl₂ (2.5 mM). Treatment was performed for 1.5 hour at room temperature with gentle stirring. NaCl was then added to a final concentration of 1M and allowed to dissolve. Next, 80 g of PEG-8000 was added to the lysate and stirred at room temperature until dissolved. The lysates were then stirred at 4° C. for 45 minutes and subsequently incubated overnight at 4° C. without stirring for precipitation. The precipitated lysates were centrifuged at 10,000×g for 15 minutes at 4° C. The supernatant was removed, and the pellets were allowed to dry at room temperature. Pellets were resuspended in 7 mL of SM Buffer (100 mM NaCl, 25 mM Tris-HCl pH 7.5, 8 mM MgSO₄). To remove the insoluble material, the resuspended pellets were centrifuged at 3000×g for 10 minutes twice, moving the supernatant to a new tube after each spin. CsCl was added to the supernatant at a rate of 0.75 g/mL. The phage library solutions were added to a step gradient of 1.4, 1.5, and 1.6 g/mL CsCl in 14 mL Ultra-Clear centrifuge tubes (Beckman Coulter #344060) and centrifuged in a SW40 Ti rotor (Beckman Coulter) at 24,000×rpm for 24 hours at 5° C. For each tube, a faint light blue band was observed between the 1.4 and 1.5 layers and extracted using a syringe and a 26-gauge needle. The purified phage libraries were then dialyzed overnight at 4° C. using Slide-A-Lyzer™ cassettes with a 10 k MWCO (Thermo Scientific #66380) in SM Buffer supplemented with 1M NaCl. Two additional rounds of dialysis were performed in normal SM Buffer for 2 hours at room temperature. After dialysis, the phage were extracted from the cassettes and filtered with a 0.22 μm PES membrane filter. Plaque assays (described above) using DH10B as a host were performed to titer the libraries.

Sequencing PhageMaP libraries: Phage genomic DNA from libraries were extracted using the Norgen Phage DNA Isolation Kit (Norgen #46800) with DNase and proteinase K treatment, following manufacturer instructions. Primers BC_2N_Twist_NGS_F and BC_4N_Twist_NGS_R were used to amplify barcodes for sequencing using the following PCR cycle settings in 20 μL reactions: 95° C. 3 minutes, (98° C. 20 seconds→65° C. 15 seconds→72° C. 15 seconds)×20 cycles, 72° C. 1 minute, and 4° C. hold. Illumina® i7 and i5 adapter sequences containing unique indices and were added after performing a second 25 μL PCR reaction with the following cycle settings: 95° C. 3 minutes, (98° C. 20 seconds→65° C. 15 seconds→72° C. 15 seconds)×15 cycles, 72° C. 1 minute, and 4° C. hold. Libraries were quantified using a Qubit™ 4 as described above. The T7 library was sequenced using a 2×150 Miseq Nano Kit (Illumina®). The Bas63 library was sequenced using a 2×250 Miseq Nano Kit (Illumina®).

Sequencing data processing and analysis: Read pairs were merged and filtered with Fastp, requiring a minimum of 90% of the read having scores above Q25. The adapter sequences were removed, and barcodes sequences were extracted using Cutadapt. The resulting sequences were further analyzed using custom Python scripts.

ddPCR for recombination efficiency estimate: Digital droplet PCR was performed to estimate abundance of recombinant population in the phage library. The estimation was calculated by first finding the percentage of phages that contain a barcode and then finding the percentage of barcodes that belong to the residual donor plasmid after phage library purification. The percentage of phages that contain a barcode was determined with a primer-probe pair where one primer-probe produces signal for the barcode insert (present in donor plasmids and recombined phage) and another primer-probe produces signal for a constant region in the phage (present in both recombined and unrecombined phage). The ratio of the former to the latter is the amount of barcode containing DNA detected relative to phage DNA. To account for the donor plasmid population, a second primer-probe pair is used where one primer-probe produces a signal for the kanamycin cassette found in the donor plasmid and another primer-probe produces a signal for the barcode insert (present in donor plasmids and recombined phage). The ratio of the former to the latter gives an estimate of the percentage of barcodes that originate from plasmid DNA. Multiplying the first ratio with the difference between 1 and the second ratio gives us an estimate of the percentage of phages with barcodes (recombinant percentage). For T7 libraries, 3 pg of DNA was used for each reaction. For Bas63, 6 pg of DNA was used. For T4, 12 pg of DNA was used. All reactions were performed in 20 μL, using ddPCR supermix for probes (no dUTP) (Biorad #1863024), 0.9 μM of each primer, and 0.25p M of each 5′ 6-FAM/ZEN/3′ IBFQ or 5′ HEX/ZEN/3′ IBFQ probe with the QX200 Droplet Digital™ PCR System (Biorad). Reactions were performed using the following PCR cycle settings: 95° C. 10 minutes, (94° C. 30 seconds→60° C. 1 minute)×40 cycles, 98° C. 10 minute, and 12° C. hold.

PhageMaP selection experiments and analysis: PhageMaP selection-Overnight cultures of the target hosts were back diluted 1:10 in LB with additional supplements if necessary. Cultures were grown until OD₆₀₀ ˜0.7-1 and aliquoted into three separate tubes to serve as replicates. The phage library was added at an MOI 0.1 to ensure comprehensive assaying of the library and single infections. All cultures were allowed to lyse at 37° C. For the laboratory E. coli strains infected by T7, cultures were collected after 1.5 hour. For the phage defense strains infected by T7, cultures which had visual lysis were collected after 3 hours. For the laboratory E. coli strains infected by Bas63, cultures were allowed to lyse overnight. For the 9 phage defense strains, including empty vector, infected by Bas63, cultures were allowed to lyse for 7 hours. For the laboratory E. coli strains grown in M9 media and infected by T4, cultures were collected after 23 hours. For the laboratory E. coli strains grown in LB media and infected by T4, cultures were collected after 5 hours. All lysates were purified using 0.22 m PES membrane filters.

Sequencing phages after selection: Three pairs of primers containing 1, 2, or 3 N offsets and a unique 3 nucleotide barcode (for demultiplexing of replicate numbers) were used to amplify barcodes from 10 μL of phages after selection with the following PCR cycle settings in 25 μL reactions: 95° C. 5 minutes, (98° C. 20 seconds→65° C. 15 seconds→72° C. 15 seconds)×20 cycles, 72° C. 1 minute, and 4° C. hold. Illumina® i7 and i5 adapter sequences containing unique indices and were added after performing a second 25 μL PCR reaction with the following cycle settings: 95° C. 3 minutes, (98° C. 20 seconds→65° C. 15 seconds→72° C. 15 seconds)×15 cycles, 72° C. 1 minute, and 4° C. hold. Libraries were quantified using a Qubit™ 4 as described above. The T7 and T4 selections were sequenced using 1×100 NextSeq™ P2 Kits (Illumina®) on a NextSeq™ 1000. The Bas63 selections were sequenced using a 2×150 NovaSeq™ Kit (Illumina®) through the University of Wisconsin Biotechnology Center Sequencing Core.

Analysis of selection data: Read1 in each sequencing run was filtered with Fastp, requiring a minimum of 90% of the read having scores above Q25. The adapter sequences were removed, and barcode sequences were extracted using Cutadapt. The resulting sequences were further analyzed using custom Python scripts. Barcodes mapping to the same sgRNA were summed. sgRNAs which do not have at least 2 reads post-selection and 15 reads pre-selection were excluded from analysis. Calculation of fitness scores were adapted from Enrich2. Log ratios (L) for total count (c) of each sgRNA (s) in the initial (i) and selected (sel) were calculated using the following formula:

$${L}_{s} = log ( \frac{{c}_{s , sel}}{{c}_{t o tal , sel}} ) - log ( \frac{{c}_{s , i}}{{c}_{t o tal , i}} ) \qquad ( 1 )$$

The standard error (SEₛ) for each log ratio was then calculated using the following formula:

$$S {E}_{s} = \sqrt{\frac{1}{{c}_{s , i}} + \frac{1}{{c}_{t o tal , i}} + \frac{1}{{c}_{s , sel}} + \frac{1}{{c}_{t o tal , sel}}} \qquad ( 2 )$$

The log ratios and standard errors for all sgRNAs of the same gene were combined into a single gene score and standard error using the Enrich2 restricted maximum likelihood estimation with 50 Fisher scoring iterations. Overlapping regions of genes and intergenic regions are scored as individual genes. These genes scores from each of the replicates were once again combined into a single composite gene score using the same restricted maximum likelihood estimation. To help compare scores across conditions, each composite gene score was Z-normalized within each condition. To identify significant genes for the phage defense screens, these Z-normalized composite gene scores and their standard errors of phage defense conditions were compared to no defense conditions using Welch's two-sided t-test. A pre-multiple testing correction significance threshold of 0.05 and post-correction threshold of 0.1 were set. Here, counters are defined as genes which are nonessential (composite gene score >0.25) in the empty vector control but become significantly more essential. Triggers are defined as genes which are essential (composite gene score <0.25) in the empty vector control but become more nonessential.

Quantifying Cas9-RecA/sfGFP recombination efficiency: Construction of donor cells—Upon successful recombination, a 100 bp deletion in T7g1.1 is replaced with 43 bp containing a 11N barcode and barcode priming sites. A gene fragment containing 50 nt homology arms, 11N barcode, J23119 promoter, sgRNA targeting T7g1.1, and BsmBI sites at the termini was PCR amplified using primers g576-Twist+2969. Primers pUC19_BB_F+pUC19_BB_R were used to amplify the pUC19 vector with homologous ends to the gene fragment. These two fragments were assembled using Gibson Assembly® and transformed using the transformation protocol outlined above. After plating, one colony was chosen after PCR and sequencing validation. This colony was used for miniprep. The resulting plasmid was transformed into E. coli DH10B Cas9-RecA or Cas9-sfGFP competent cells.

Testing recombination in Cas9-RecA and Cas9-sfGFP cells: Overnight cultures of Cas9-RecA and Cas9-sfGFP cells were back-diluted and grown to OD₆₀₀ 0.3 at 30° C. To cells that require induction, anhydrotetracycline was added to a final concentration of 1 μM. Uninduced and induced cells were grown for 1 hour at 30° C. and subsequently diluted back down to OD₆₀₀ 0.3. Wildtype T7 phage was added to 1 mL of each culture in triplicate and allowed to infect for 1.5 hour. Lysates were spun down at 16,000×g for 1 minute prior to filtering the supernatant using 0.22 μm PES membranes.

Sequencing Cas9-RecA and Cas9-sfGFP lysates: To generate sequencing amplicons for both libraries, 1 μL of phage lysate was used as template for a 25 μL PCR reaction with primer pairs 576_NGS_4N_NGS_F+576_NGS_4N_NGS_R containing 4N offsets and the following PCR cycle settings: 95° C. 5 minutes, (98° C. 20 seconds→60° C. 15 seconds→72° C. 15 seconds)×12 cycles, 72° C. 1 minute, and 4° C. hold. Illumina® i7 and i5 adapter sequences containing unique indices and were added after performing a second 25 μL PCR reaction with the following cycle settings: 95° C. 3 minutes, (98° C. 20 seconds→465° C. 15 seconds→72° C. 15 seconds)×8 cycles, 72° C. 1 minute, and 4° C. hold. PCR products were purified with the E.Z.N.A.® Cycle Pure Kit and quantified using the Qubit™ 4 as described above. The purified products were pooled and sequenced on an Illumina® MiSeq with a 2×250 Miseq Reagent v2 kit.

Sequencing data processing and analysis: Read pairs were merged and filtered with Fastp, requiring a minimum of 90% of the read having scores above Q25. The resulting sequences were further analyzed using custom Python scripts. Merged reads containing the known inserted barcode (GTTTAGGCTGT; SEQ ID NO: 1) and a consensus sequence (GATTTAAATTAAAGAATTAC; SEQ ID NO: 2) found in both recombined and unrecombined phages were quantified. Percent recombinant was calculated by finding the ratio of sequences containing the barcode and sequences containing the consensus sequence.

Validation of counter and triggers: Generating T7 counter knockouts—To determine if knockout of specific genes reduces the fitness of T7 relative to WT, efficiency of plating measurements were made between wildtype and knockout phages on relevant phage defense backgrounds. Amplicons were generated from the linear wildtype genome with homology at the termini to neighboring amplicons. Amplicons were assembled using a 5-part Gibson Assembly® such that the central ˜80% of the target gene is deleted. 0.08 to 0.1 pmol of each amplicon was used in each reaction. T7g0.3 knockout was previously constructed using a 6-part Gibson Assembly® where a 173 bp insert is inserted into position 212 of the gene. Reactions were dialyzed and transformed into DH10B competent cells. After transformation, transformants were plated with 4 mL top agar on LB plates and allowed to incubate at 37° C. overnight. Single plaques were PCR verified before creating stocks using DH10B as the host.

Generating Bas63 counter knockouts: To determine if knockout of specific genes reduces the fitness of Bas63 relative to WT, efficiency of plating measurements were made between wildtype and knockout phages on relevant phage defense backgrounds. The knockouts were synthesized using Cas9-RecA-mediated homologous recombination and were designed such that the central ˜80% of the target gene is deleted. Gene fragments (Twist) containing 100 nt homology arms, J23119 promoter, and sgRNA targeting the relevant position of the genome were inserted into pUC19-Donor-BsmBI with Golden Gate Assembly (BsmBI). Transformation and plating procedures were performed as described above. Single colonies were isolated, sequence verified, grown up for miniprep, and then transformed into DH10B Cas9-RecA competent cells. Colonies were grown overnight at 30° C. Overnight cultures of these strains containing donor plasmid and Cas9-RecA were back diluted 2-fold in LB-Kan-Spec and induced with anhydrotetracycline to a final concentration of 1 μM for 30 minutes at 30° C. To perform recombination, 300 μL of induced culture and 10⁴-10⁵ Bas63 phages were mixed into 0.7% top agar containing kanamycin, spectinomycin, and anhydrotetracycline. The top agar mix was poured onto LB plates. Plates were incubated at 37° C. overnight. Successful plaques were verified with PCR prior to performing further experiments.

Efficiency of plating assays: To identify optimal dilutions to use for plaque assays, a spot plate assay was first performed using serial dilutions of the phages and spotting on a lawn of relevant hosts in 0.7% top agar. Plaque assays were performed in triplicate by adding 300 μL anti-phage defense host (or empty vector) and the optimal dilution of phage to 4 mL of 0.7% top agar supplemented with chloramphenicol (25 μg/mL) and pouring the mix into LB plates. For gene complementation hosts, kanamycin (50 μg/mL) and 0.1% L-arabinose (w/v) was added to the top agar prior to pouring. Plates were counted after an overnight incubation at 37° C. Plates must have between 10-400 plaques to be included in further analysis. T7 genes 4.3 and 4.5 were too toxic when induced, thus complementation was performed from the leakiness of the uninduced cells. For validations of counters, the average titer of each phage on the control strain was first determined (3 technical replicates). Titers of the phage on the defense strain were then obtained in triplicate. EOP values were calculated as the average ratio of each replicate on the defense strain with the previously calculated average titer on the control strain.

Constructing trigger plasmids: Candidate trigger genes were amplified from the wildtype genome via PCR. Amplicon ends contain homology to the vector backbone ends amplified from pSM103 with primers pSM103-BB-R+1517. Constructs were assembled with Gibson Assembly as described above. The result is an arabinose-inducible expression plasmid for each candidate trigger gene. Plasmids were sequenced (Plasmid saurus) prior to transformation into phage defense competent cells prepared the same way as described above. Transformants were recovered in SOC and plated on LB-Kan-Cam plates supplemented with 0.2% D-glucose (w/v) to repress expression of trigger genes.

Lysis area quantification: Approximately 2E8 CFU of Empty vector, ppl, ppl+sfGFP, or ppl+gp130 cells were added to 2 mL LB top agar, mixed, and poured on top of LB plates supplemented with 0.1% L-arabinose and the necessary antibiotics (Cm25 for Empty vector and ppl, Cm25+Kan50 for ppl+sfGFP and ppl+gp130). Plates were allowed to cool for 1 hour. Then ˜5E6 PFU of wildtype T7 or T7Δ4.3::gp130 were applied to the plate as a 3 L droplet. Plates were incubated at 37° C. for 68 hours and then removed for plaque diameter measurement.

Inosine Quantification: Overnight DH10B cultures of Empty vector+pSM103-T7gp5.7 and RADAR2+pSM103-T7gp5.7 were backdiluted 1:10 in 6 mL of LB supplemented with kanamycin and chloramphenicol and grown until OD₆₀₀ 0.6. T7 gp5.7 expression was then either repressed with 0.2% D-glucose (w/v) or induced with 0.1% L-arabinose (w/v) for 1 hr at 37° C. The cultures were spun down at 3280×g for 10 min at 4° C. to remove the supernatant. The pellets were resuspended in 600 μL of 100 mM Tris-HCl pH7.5 and frozen in liquid nitrogen. To lyse the cells, the frozen cells were thawed at room temperature and added to ˜500 L 0.1 mm glass beads in a cryovial. Cells were homogenized using a VWR 4-Place Mini Bead Mill Homogenizer at speed 5 for 2×50 sec at 4° C. The cell lysates were spun down at 15,000×g for 10 min at 4° C. to remove cellular debris. The supernatants were removed, added to a Pierce™ Protein Concentrator PES, 3K MWCO (Thermo Scientific #88512), and spun at 12,000×g for 45 min at 4° C. The filtrate in the collection tube was kept for further processing. Because the Inosine Quantification Assay Kit (Fluorometric) (Abcam #abl26286) does not have specificity towards phosphorylated forms of inosine, a dephosphorylation step using Quick CIP (NEB #M0525) was performed. Dephosphorylation was performed at 37° C. for 2 hr using the filtered lysates in 100 μL reactions, scaled up according to manufacturer instructions. The phosphatase was deactivated at 80° C. for 2 min prior to proceeding to the next step. For the fluorometric quantification assay, 30 μL of the dephosphorylated samples was mixed with 20 μL reaction buffer and processed in 100 L total reaction volume using black 96-well plates, as described by the manufacturer. Fluorescence values were measured using a Synergy HTX Multi-Mode 96-well plate reader. Inosine levels were obtained by comparing fluorescence values to a provided inosine standard curve.

### Example 1: PhageMaP Creates Barcoded Genome-Wide Knockout Libraries in Phages

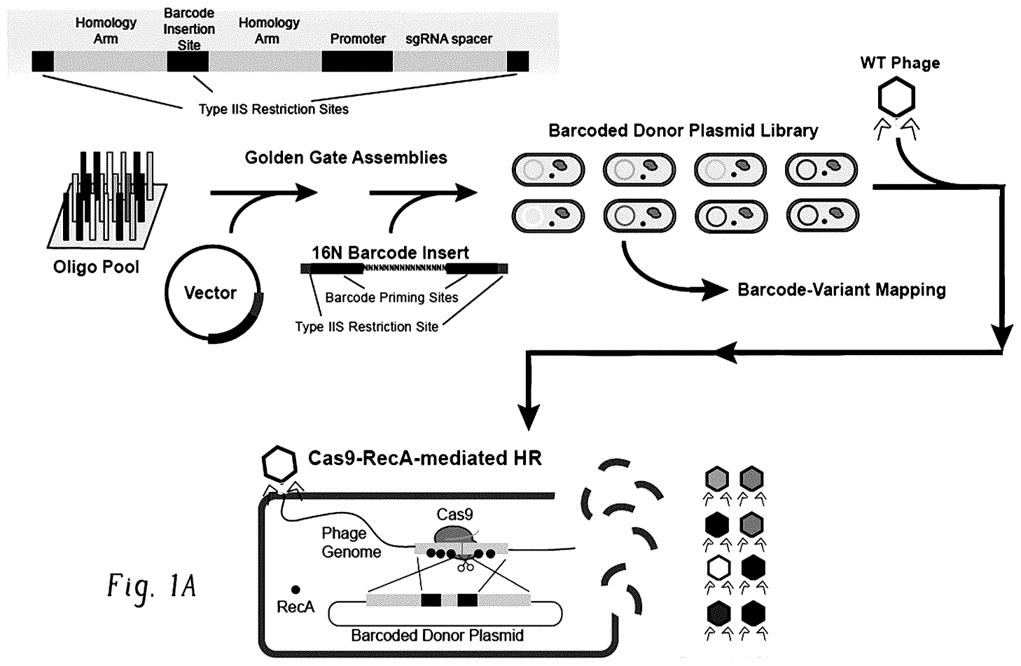

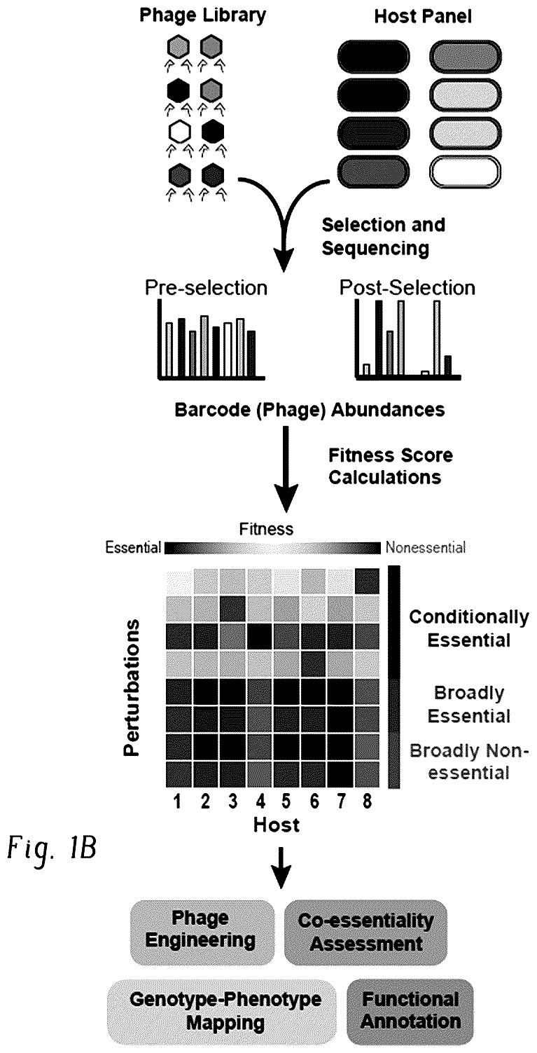

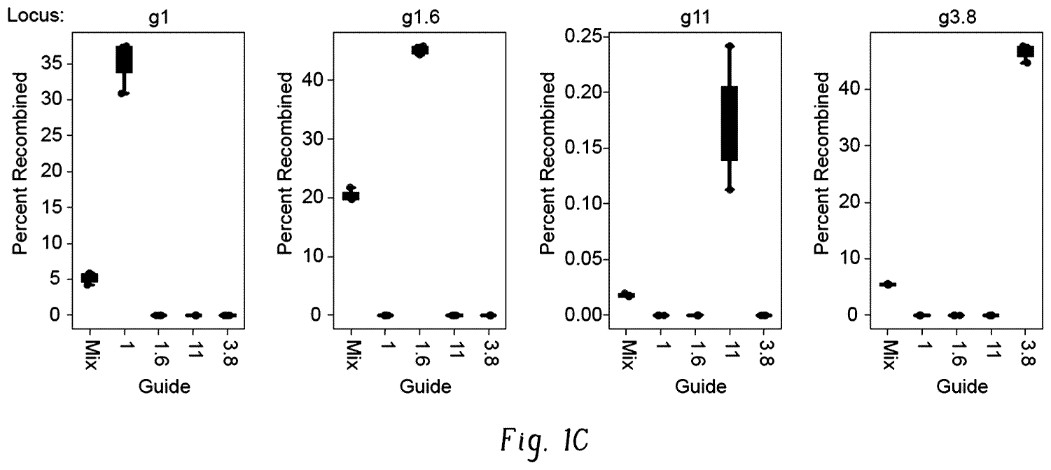

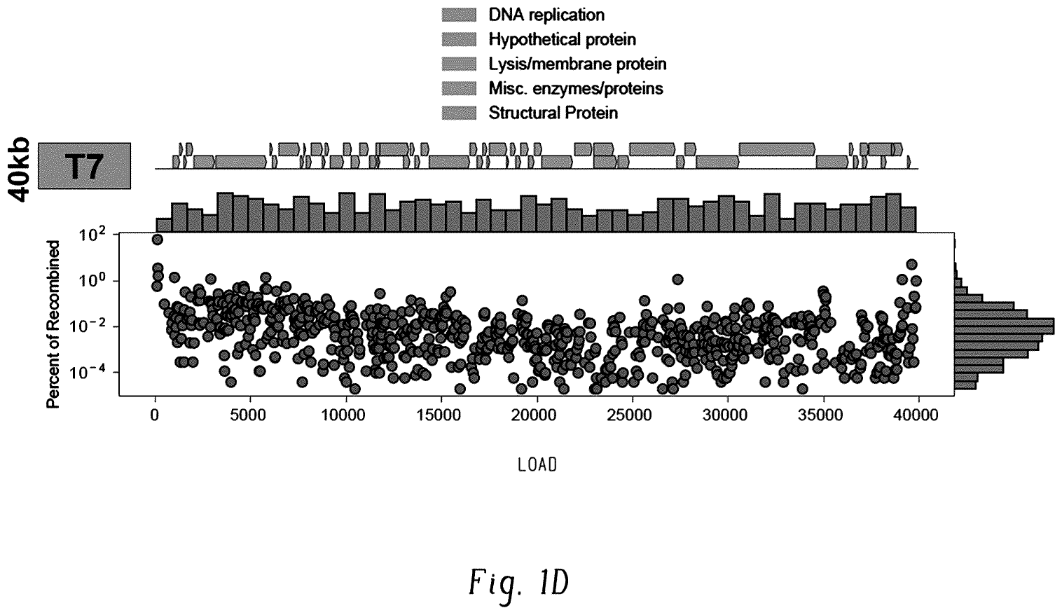

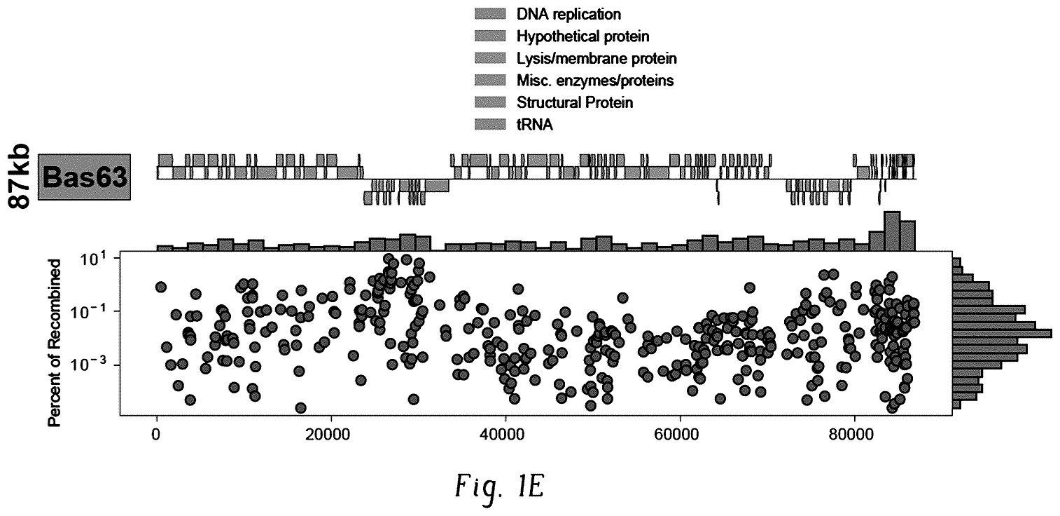

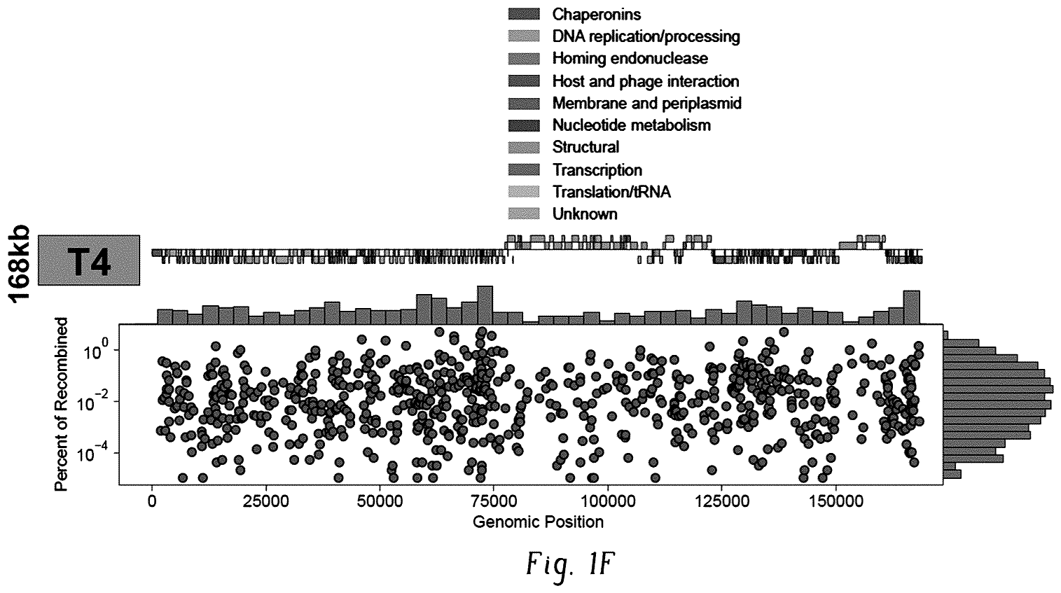

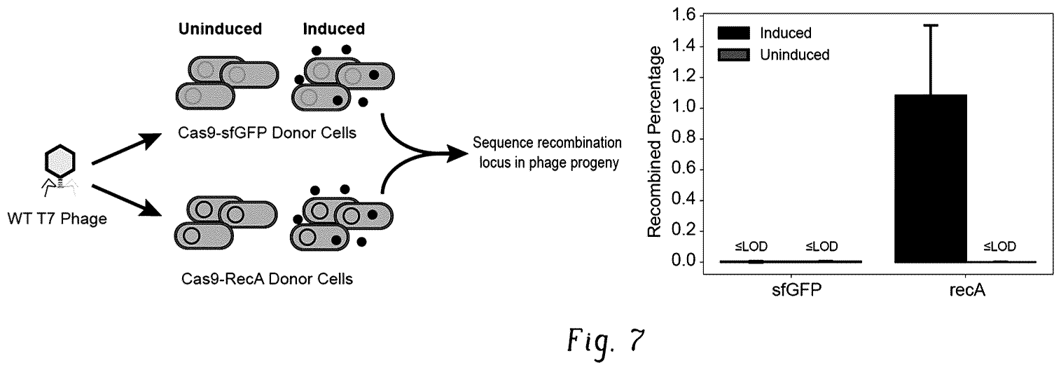

PhageMaP is carried out in three steps: (1) pooled barcoded donor library synthesis and mapping, (2) barcode integration using HR (FIG. 1A), and (3) deep sequencing to obtain quantitative gene essentiality mapping across conditions (FIG. 1B). Each donor library member contains unique sgRNAs, barcodes, and donor sequences and is used as the repair template for HR after Cas9 cleavage to generate a barcoded loss-of-function phage variant (FIG. 1A). RecA was added to boost recombination efficiency (FIG. 7).

After recombination, the resulting barcoded phage knockout library is challenged on susceptible hosts. Pre- and post-selection barcode counts are used to calculate fitness scores and assess conditional essentiality across tested hosts (FIG. 1B). Mutants in essential regions should deplete and have lower scores post-selection, while those in nonessential regions persist and have higher scores. Because the same pre-selection phage library is used for each condition, biases that may arise from using different starting libraries of variants are accounted for.

To validate that PhageMaP generates insertions at targeted loci, the recombination efficiencies were assessed for four T7 loci individually and in pooled format. Two essential loci, g1 (T7 RNA polymerase) and g11 (tail tubular protein), and two nonessential loci encoding hypothetical proteins, g1.6 and g3.8, were tested. When targeted individually, efficiencies are greatest with the on-target sgRNAs (0.1-48%), and no recombinants were detected with the other off-target sgRNAs (FIG. 1C). Essential gene knockouts are possible if the protein is synthesized before Cas9 restriction. For instance, ˜35% recombination of g1 was observed even though T7 RNA polymerase is essential (FIG. 1C). In pooled format, high fidelity recombination occurred at targeted loci albeit at a lower per-gene frequency (0.02-22%) (FIG. 1C). Thus, barcodes can be efficiently and precisely inserted into targeted loci of both essential and nonessential genes in parallel.

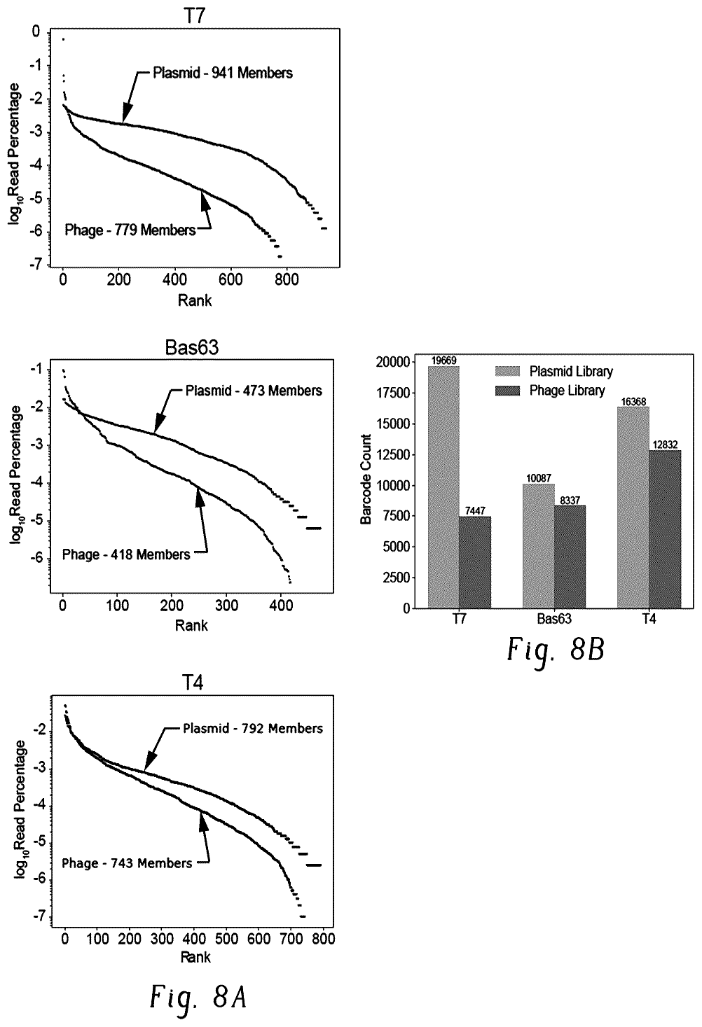

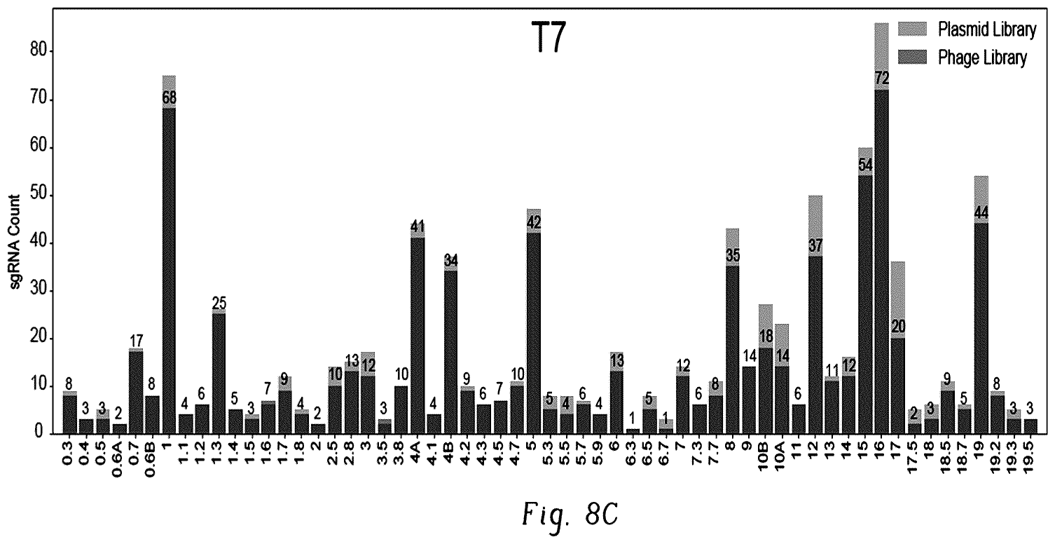

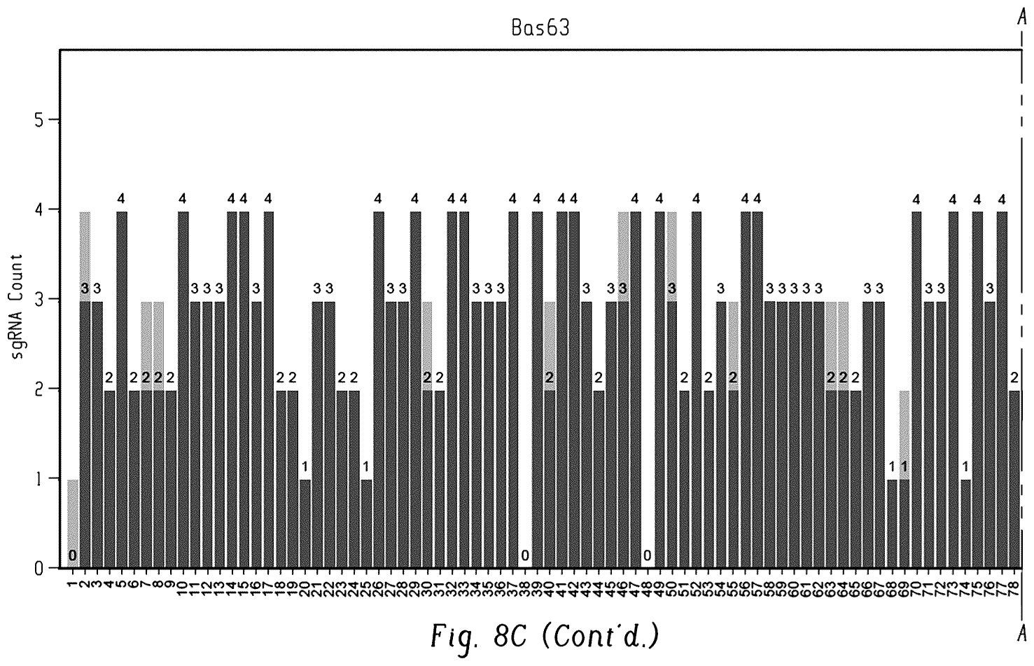

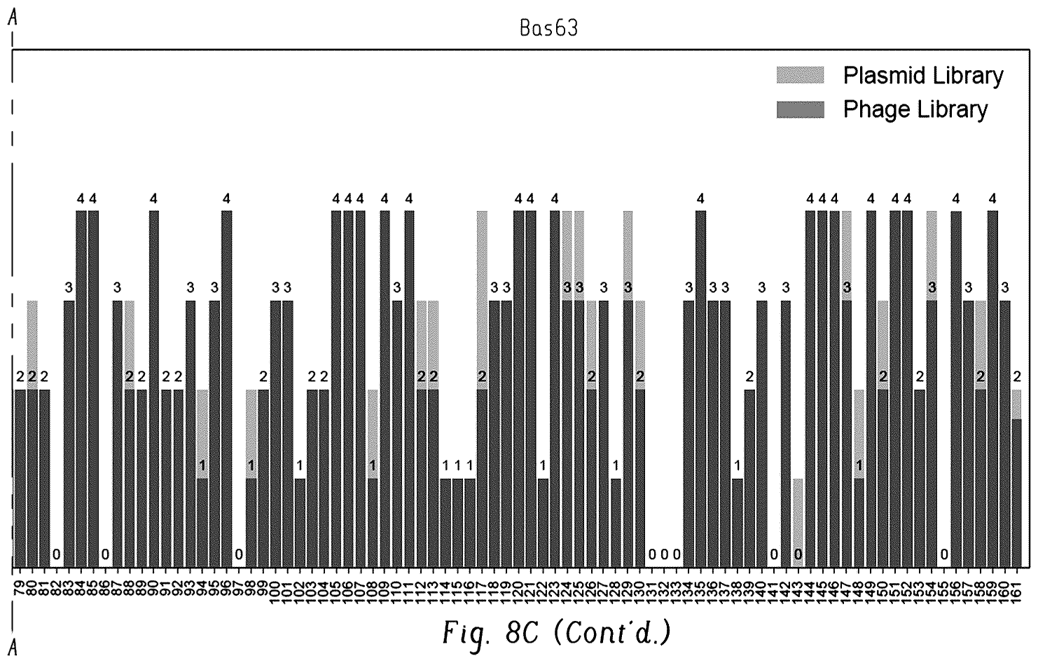

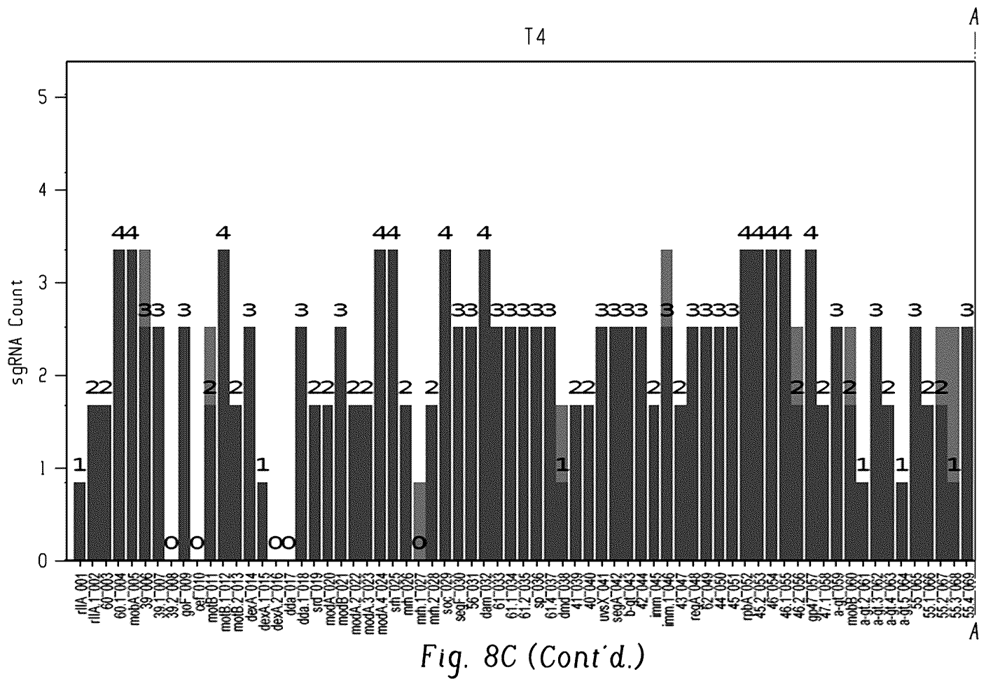

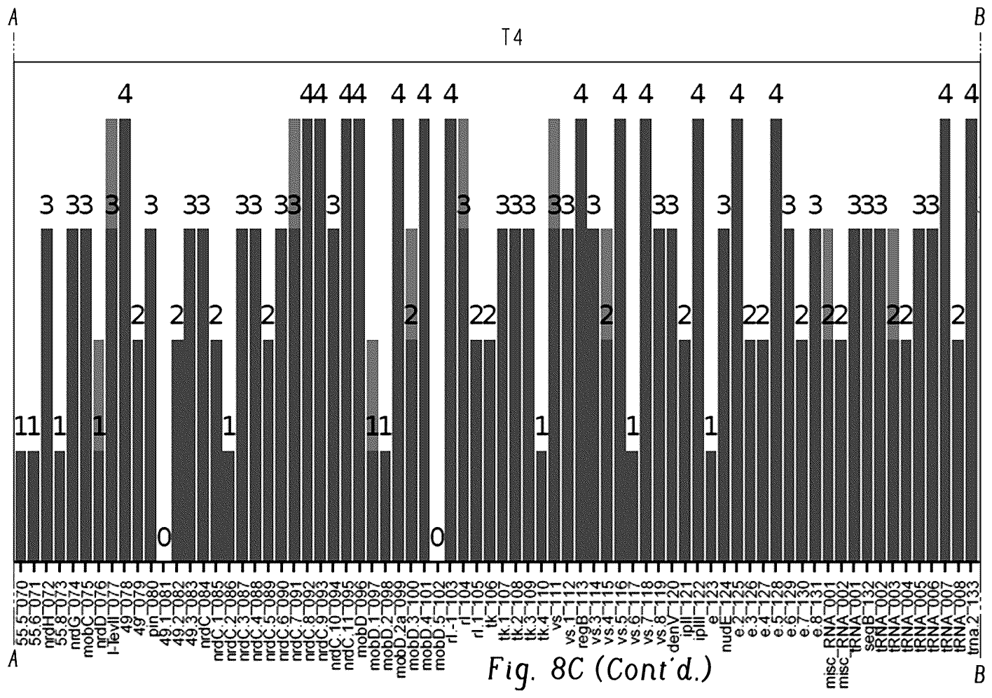

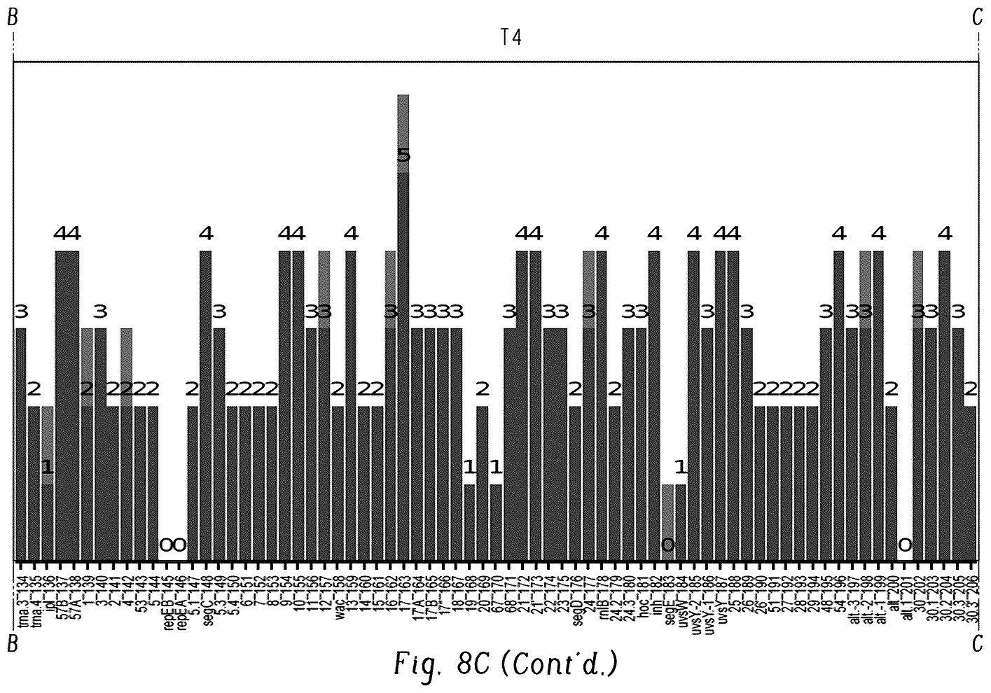

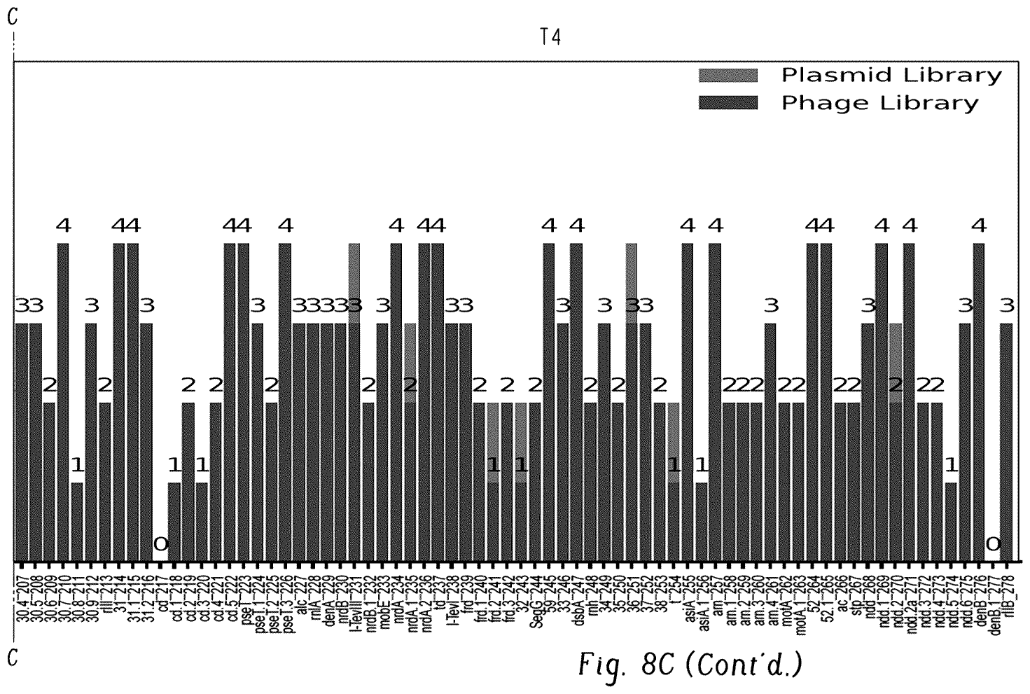

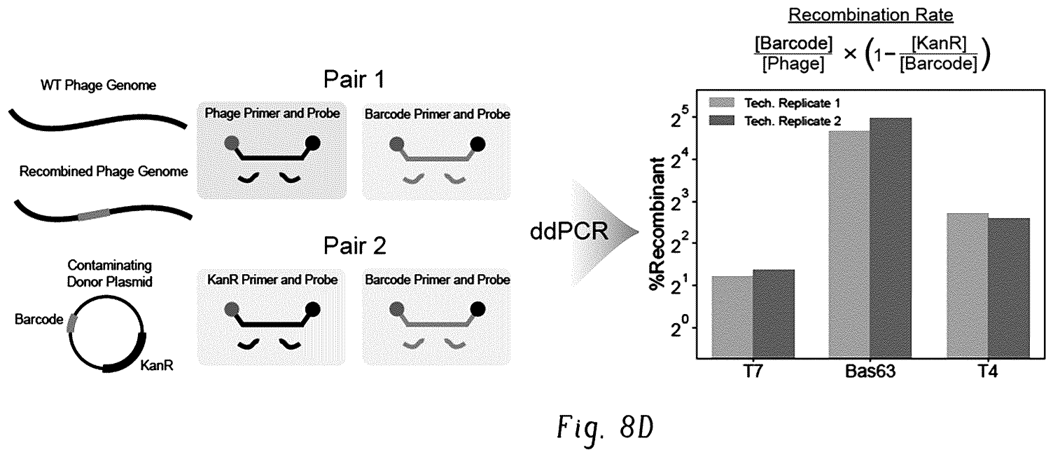

Genome-wide libraries were generated in T7 which has a 40 kb genome with about 60 genes (FIG. 1D). This donor library has a theoretical library size of 1084 sgRNAs encompassing both genic and intergenic regions. After assembly, the barcoded plasmid library contained 941 sgRNAs (85.8% theoretical) (FIG. 8A). Variants were lost likely due to low initial abundance or toxicity. The barcoded phage library dropped to 779 sgRNAs (71.9% theoretical) after recombination (FIG. 8A). After sequencing the barcoded phage library, 7447 unique barcodes were recovered (˜9-10 barcodes per sgRNA on average) and all 60 genes were represented in the library (FIG. 8B-C). The percentage of recombinants is ˜2% (FIG. 8D). Significant skew in the phage library toward the termini of the phage genome was observed which is likely due to the repeats in this region promoting recombination (FIG. 1D). To circumvent this issue in subsequent libraries, intergenic regions were omitted.

PhageMaP libraries were also created in Bas63, a non-model myovirus distinct from T7 (FIG. 1D). Bas63 is an 87 kb phage encoding 161 genes. To facilitate uniform sgRNA distribution per gene, 2-4 sgRNAs were selected for each gene to create a theoretical plasmid library with 627 sgRNAs. Following donor library construction, 473 sgRNAs were present (75.4%) (FIG. 8A). Upon recombination, 8337 unique barcodes were obtained (˜20 per sgRNA on average) for 418 sgRNAs (66.7%) and 149 out of 161 genes were recovered (FIG. 8B-C). The Bas63 recombination percentage (˜30%) far exceeded that of T7 (˜2%) (FIG. 8D). An explanation for this is the absence of a RecBCD inhibitor in Bas63 which can impair ssDNA creation for DNA repair through HR. The Bas63 library is significantly less biased than the T7 library towards specific variants, with no phage variant representing >10% of the total mapped barcodes. Without being held to theory, this is likely due to the removal of highly recombinogenic intergenic regions from the library (FIG. 1D).

To further demonstrate the generalizability and scalability of PhageMaP, a genome-scaled library for T4, a modified 168 kb Myoviridae coliphage encoding ˜300 genes, was also created. 2-4 sgRNAs were selected for each gene to create a theoretical plasmid library with 1108 sgRNAs covering all but three genes (repEA, repEB, and DenB.1) with viable sgRNAs. After construction of the donor plasmid library, 792 sgRNAs were present (71.5%) (FIG. 8A). Upon recombination, 12,832 unique barcodes (˜17 per sgRNA on average) for 743 sgRNAs (67.1%) were obtained, and 275 out of 288 genes were represented (FIGS. 8B-C). The library was ˜6% recombinant (FIG. 8D). No phage variant made up more than 5.1% of the total mapped barcodes (FIG. 1D). Like in Bas63, there was significant loss of donors/sgRNAs after assembly of the plasmid libraries likely due to toxicity of donor sequences. To obtain more comprehensive libraries, a larger number of sgRNAs per gene can be designed.

In summary, PhageMaP comprehensively and precisely generates disruptions across the phage genome in regions that are either essential or nonessential and genic or intergenic for both model (T7/T4) and non-model (Bas63) phages.

### Example 2: PhageMaP Recapitulates Established Biology and Uncovers Novel Insights in a Model Phage

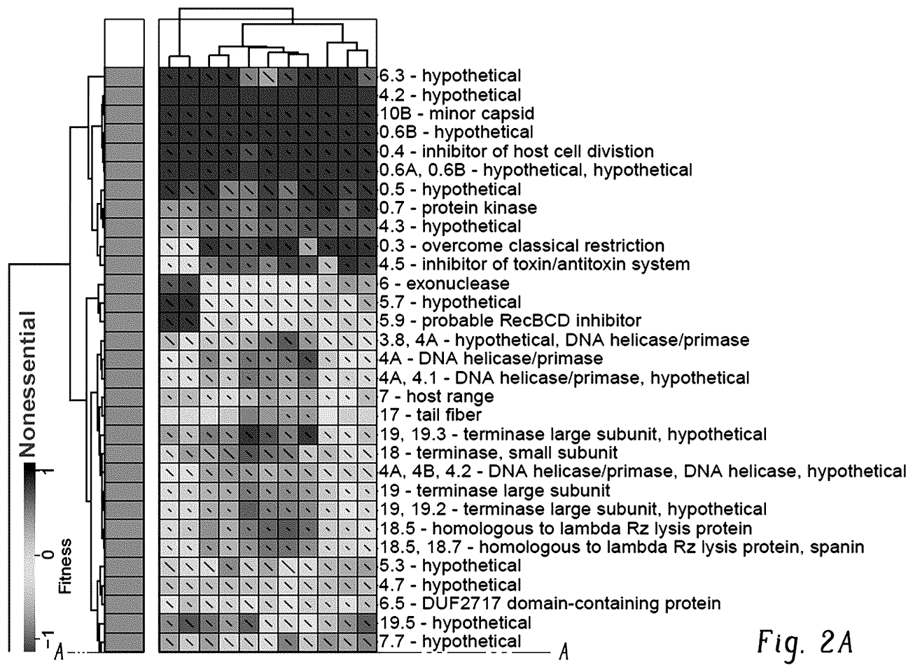

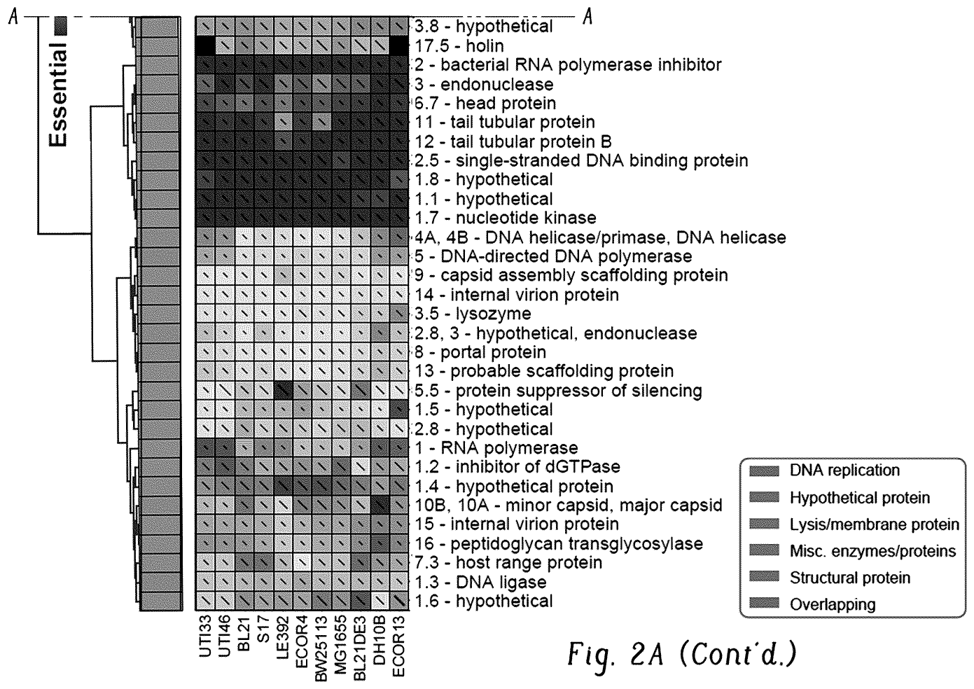

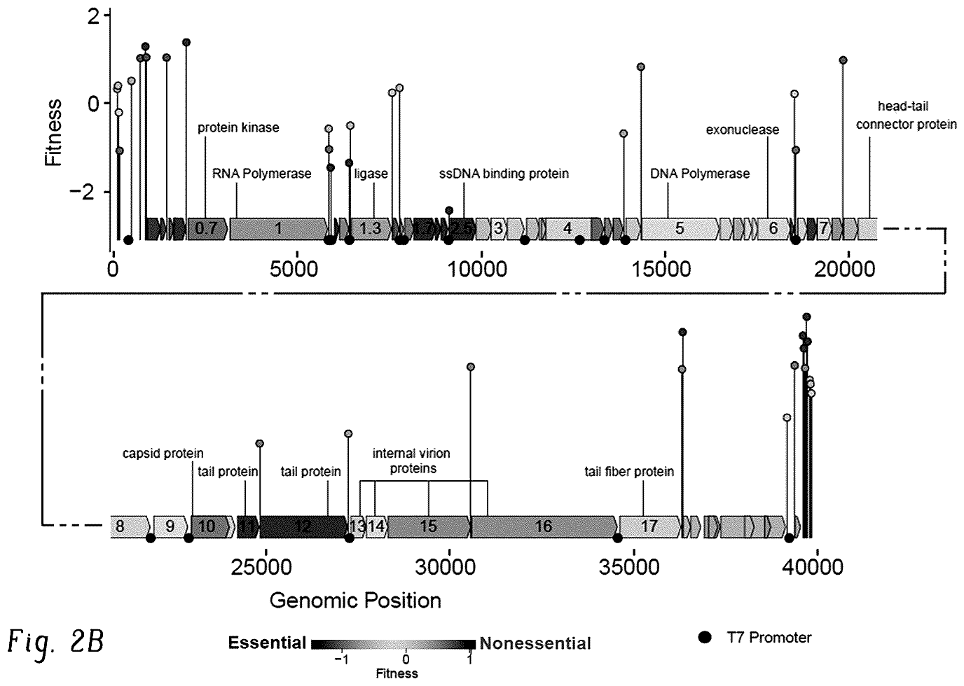

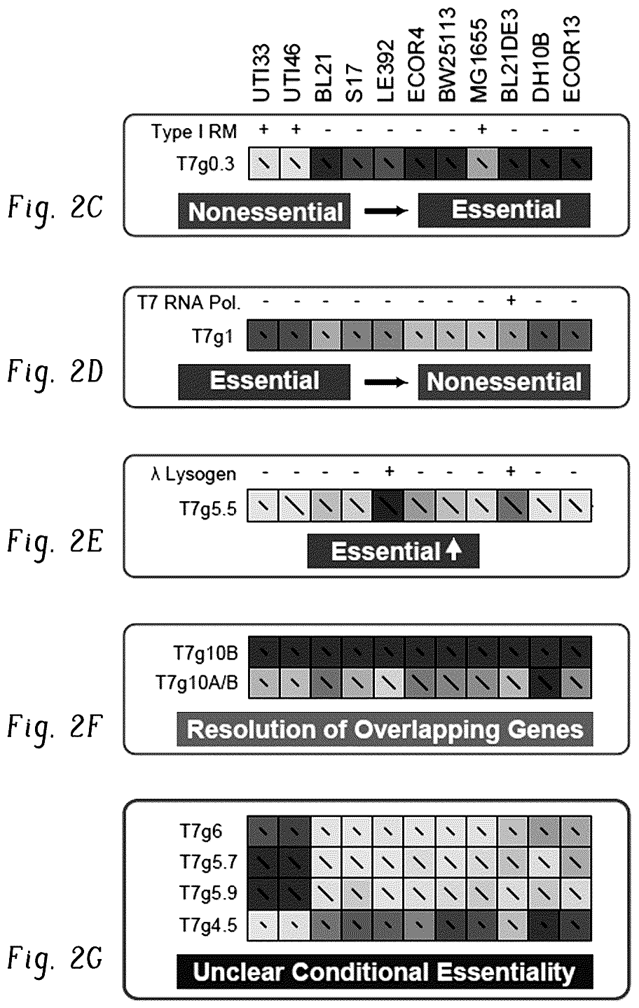

T7 was selected because its well-documented biology will help validate screen results and challenge PhageMaP to uncover novel insights for a thoroughly researched phage. The barcoded phage library was screened by infecting hosts and quantitatively scoring variants post-infection. The T7 library was applied on eleven E. coli strains: seven common laboratory strains (BL21, BL21DE3, BW25113, MG1655, DH10B, 517-1, and LE392), two clinical isolate strains from urinary tract infections (UTI33 and UTI46), and two antibiotic-resistant strains from the ECOR panel (ECOR4 and ECOR13). Across the eleven hosts, 682 fitness measurements were obtained for T7 genes (FIG. 2A). Fitness scores were highly correlated among replicates (Pearson's r: 0.90-0.98), indicating robust and reproducible barcode readouts.

To enhance the robustness of these findings, a reliability metric was implemented to assess screen performance across conditions. Based on the premise that structural genes are broadly essential for phage viability, this metric benchmarks dataset quality and informs interpretation of gene essentiality. Specifically, it was required that the third quartile of fitness scores for structural genes (3Q) was lower than the median fitness score of all other genes (MED). The 3Q threshold balances stringency while accounting for occasional nonessential structural genes. Because the T7 dataset met this threshold (3Q=−0.12; MED=0.13), results should be reliable. As structural genes can be accurately identified bioinformatically, this assessment is applicable to minimally annotated phages.

Unsupervised clustering of the normalized scores was performed across all strains to group genes based on their gene essentiality profiles (FIG. 2A). Highly nonessential and essential regions each comprise about 15% of total T7 genes, while other genes are distributed throughout a continuous essentiality scale (FIG. 2A). The two major, equally sized clades suggest half of the genes in T7 are essential (FIG. 2A). Overlaying essentiality profiles with the T7 genome map revealed co-localization of genes with similar essentiality (FIG. 2B). For instance, essential genes g1 to g2.5 were grouped, while nonessential early genes up to g0.7 were grouped (FIG. 2B). Evolutionarily, this grouping is likely preserved to maintain synteny of T7 genes with coordinated or dependent functions.

PhageMaP results align with established T7 biology and controls. Genes involved in host takeover, nucleotide metabolism, and DNA replication (1.1-1.8, 3, 3.5, 4A/B, 5, 5.5, and 6) and structural proteins (6.7, 7.3, 9, 10A/B,11, 12, 13, 14, 15, and 16) are clustered together in clades with high essentiality (FIG. 2A). Highly dispensable early genes (0.3, 0.4, 0.5, 0.6A/B, and 0.7), hypothetical genes (4.2, 4.3, and 4.7), and gene 4.5 (inhibitor of toxin-antitoxin system) are clustered in groups with low essentiality. Gene 0.3, encoding ocr which protects phage DNA from restriction-modification (RM) systems, is more essential for strains UTI33, UTI46, and MG1655, where Type I RM systems are intact (FIG. 2C). Gene 1 (T7 RNA polymerase) is less essential in BL21DE3, a strain that contains endogenous T7 RNA Polymerase which can complement the loss of this crucial enzyme in T7 (FIG. 2D). Gene 5.5, which enables growth on lambda lysogens, is more essential in LE392 and BL21DE3, the two strains containing a lambda prophage (FIG. 2E). PhageMaP also resolves overlapping genes. For instance, g10A (major capsid protein) fully overlaps with g10B (minor capsid protein), which has a 53-amino-acid extension beyond g10A's stop codon. Loss of g10B doesn't affect T7 viability provided g10A is present. The high fitness scores in g10B but not the overlapping g10A/B regions support this (FIG. 2F). These results demonstrate that PhageMaP produces biologically meaningful gene essentiality profiles.

PhageMaP enables interrogation of intergenic regions in ways CRISPRi screens, which rely on transcription or translation, cannot. 364 fitness measurements were obtained for intergenic regions in T7 across eleven strains. Seven variants located within DNA synthesis and class III gene clusters were absent in the pre-selection phage library, likely due to essential roles in regulating nearby essential genes. Regions near or at T7 promoters driving expression of essential genes (e.g. g2.5) are essential (FIG. 2B). Each of the three E. coli promoters which drive expression of the early genes is nonessential individually, possibly due to functional redundancy (FIG. 2B). Most loci at the genome ends of T7 appear nonessential. The two exceptions are positions 142 and 162, near the end of the left terminal repeat (FIG. 2B). Because the termini play roles in DNA replication, concatenation, and packaging, these positions are likely critical. Overall, intergenic essentiality aligns with genomic context.

Some genes score differently across the tested strains, indicative of conditional essentiality. For example, genes 6 (exonuclease), 5.7 (hypothetical protein), and 5.9 (RecBCD inhibitor) are less essential in UTI strains, which contain two intact prophages that may compensate for their absence (FIG. 2G). Conversely, g4.5 is more essential in the UTI strains which express the CcdAB toxin/antitoxin (TA) system. Because gp4.5 inhibits the antiviral sanaAT TA system through lon protease inhibition, it is hypothesized that gp4.5 may inhibit the CcdAB TA system whose activity is also controlled by lon-dependent proteolysis.

Taken together, PhageMaP creates a comprehensive genotype-phenotype map of T7, encompassing both protein coding and non-coding regions, across different hosts. The method recapitulates known facets of T7 biology acquired through decades of research and reproduce general gene essentiality patterns identified in previous screens. Thus, PhageMaP is a useful discovery tool to identify genes with novel functions in certain conditions for further characterization.

### Example 3: PhageMaP Creates Gene Essentiality Profiles for a Non-Model Phage

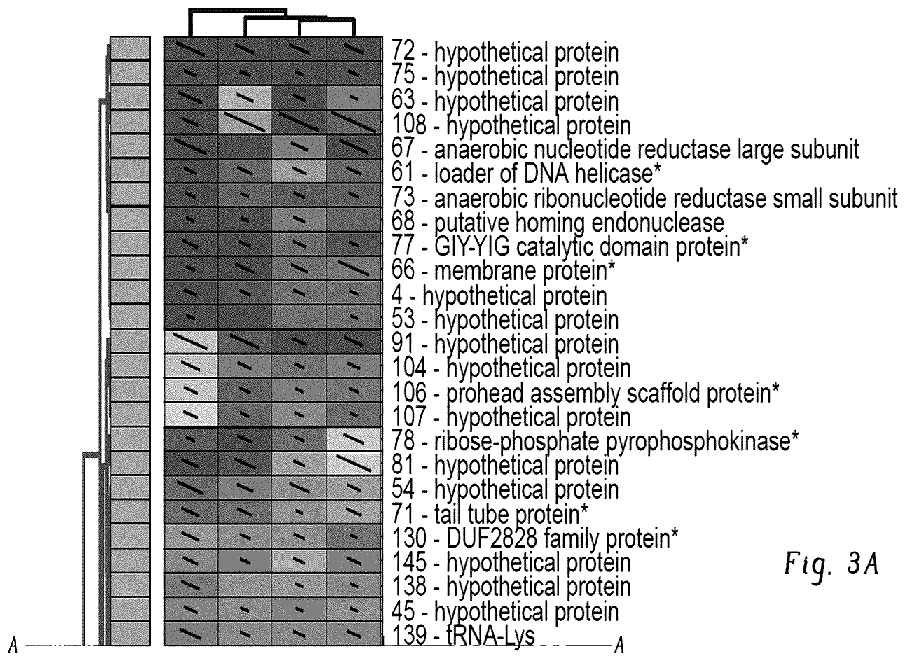

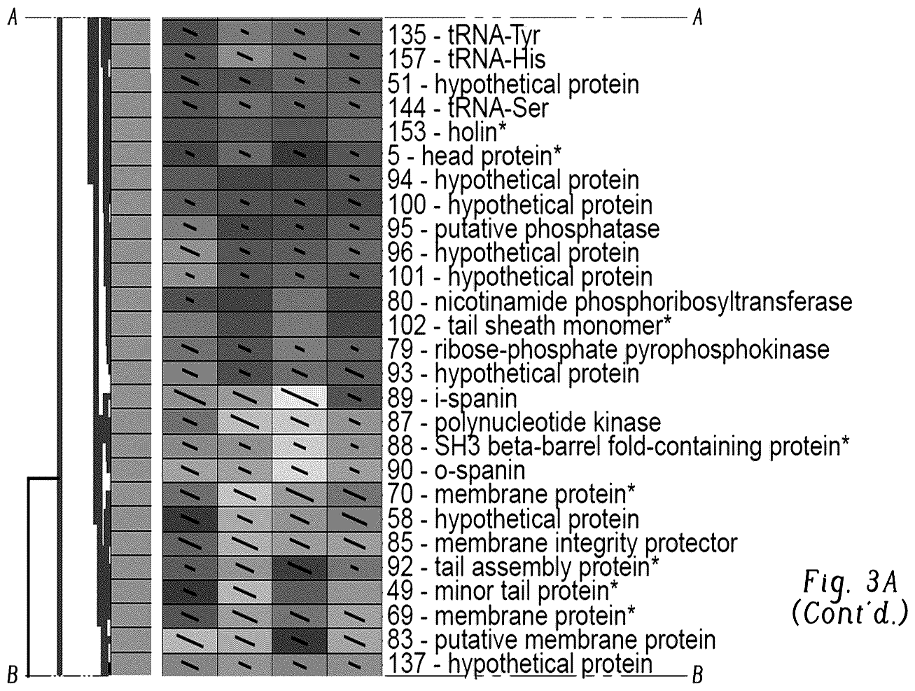

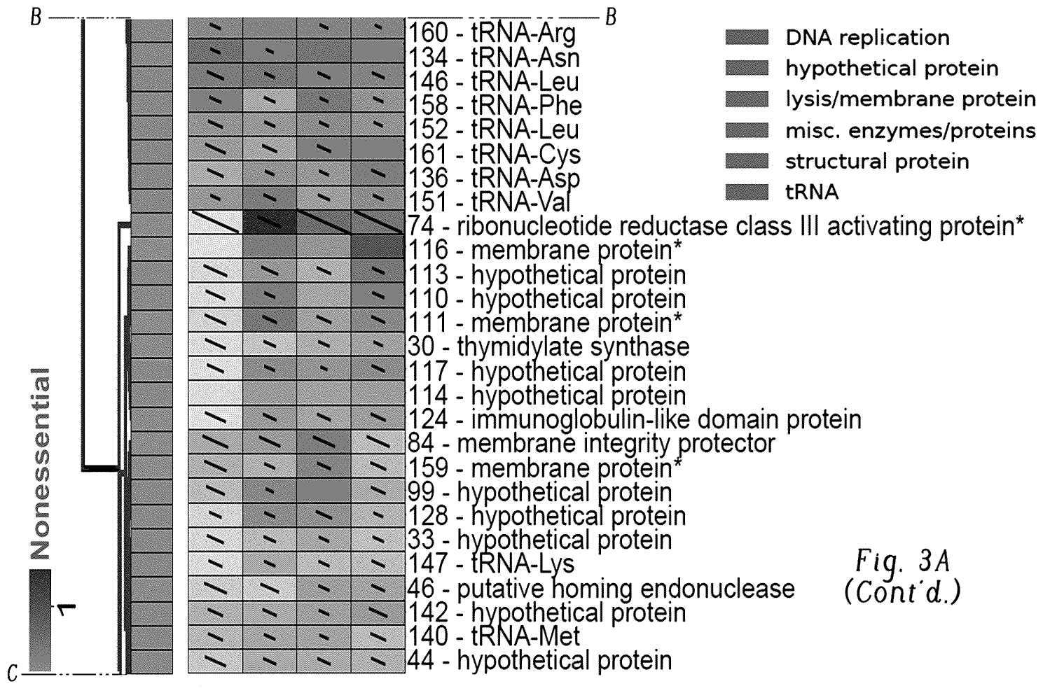

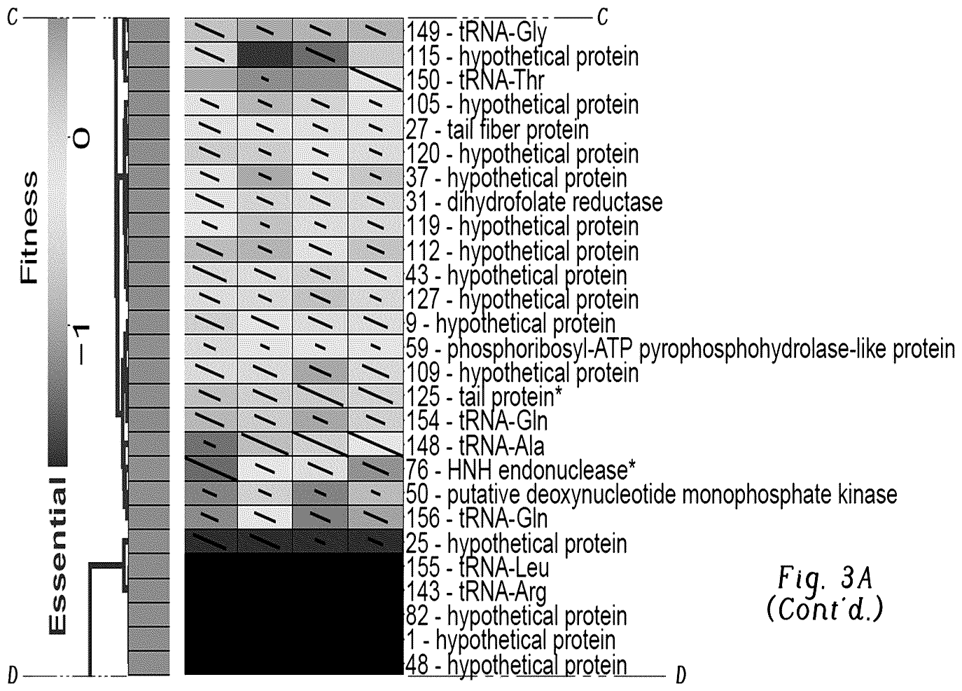

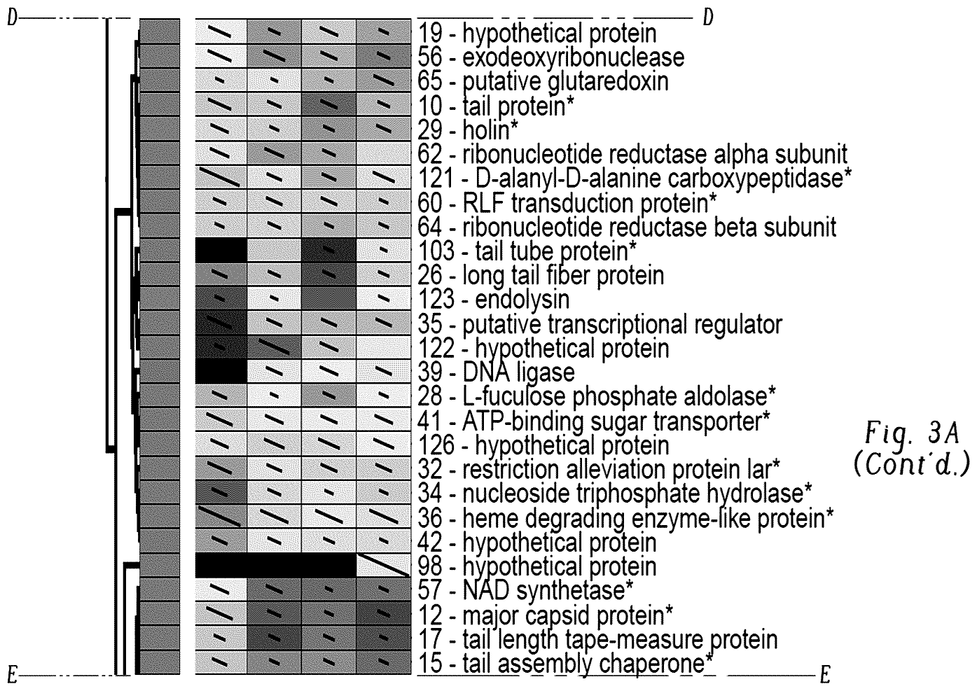

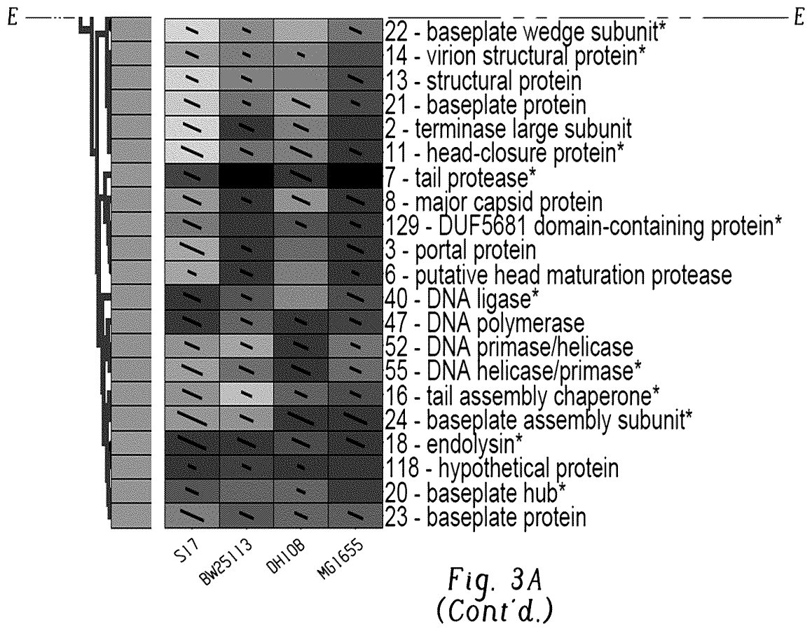

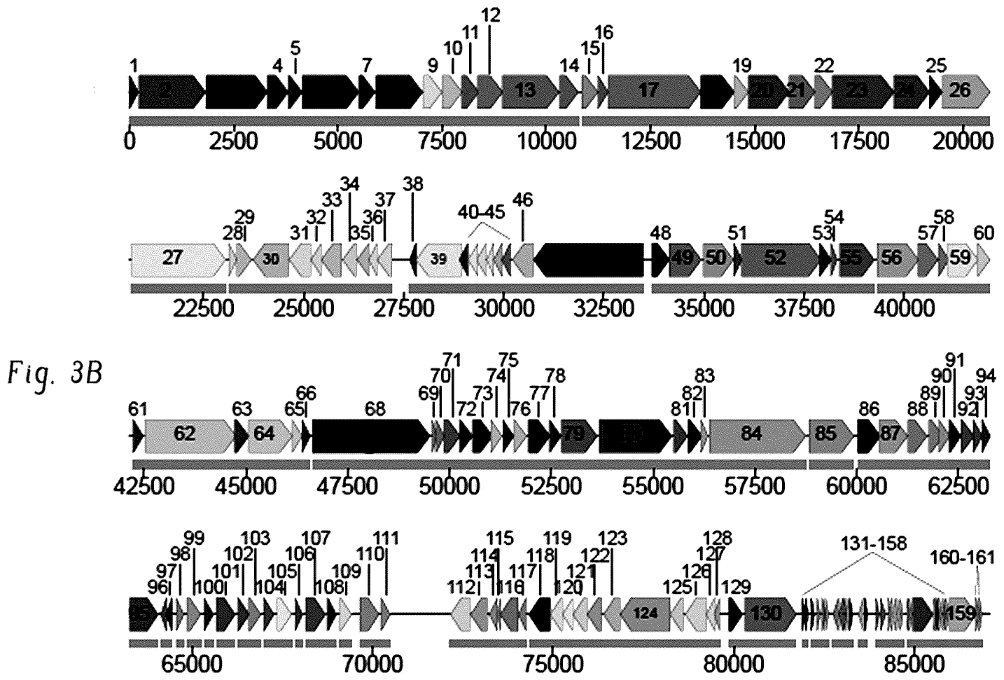

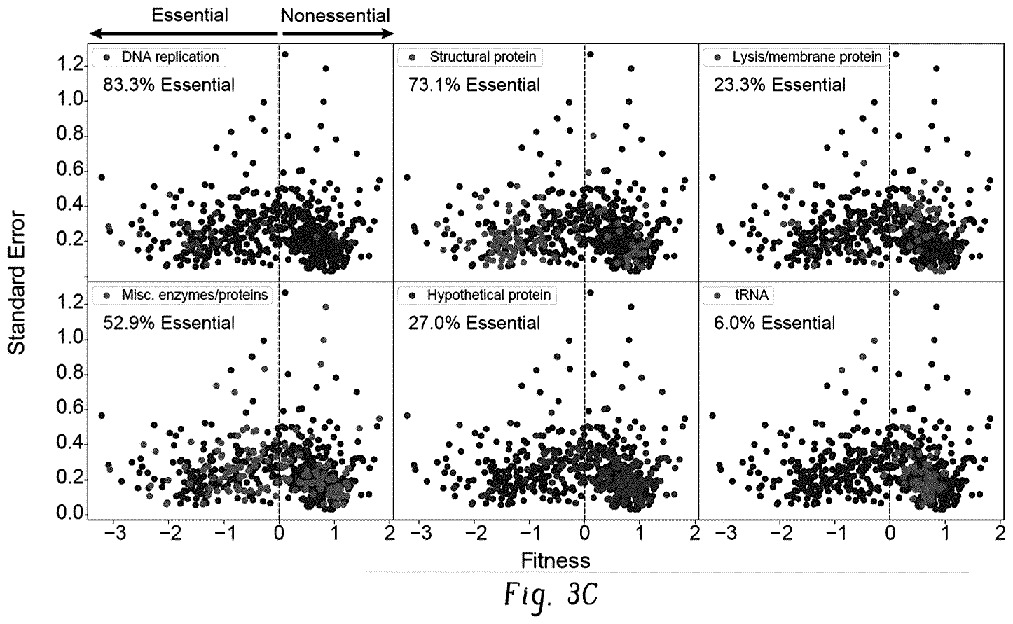

To demonstrate generalizability and scalability of our approach, PhageMaP was used to characterize gene essentiality in Bas63, a recently isolated natural Myovirus phage belonging to the Felixounavirus subfamily. Bas63 has more than double the genome size and almost triple the gene content of T7. Across four susceptible E. coli hosts, 593 fitness measurements for 149 genes were obtained with strong correlation (Pearson's r: 0.80-0.89) (FIG. 3A). Fitness scores across conditions satisfied the reliability criterion (3Q=0.11; MED=0.45). Unsupervised clustering of the genes based on their scores resulted in a distinct clade belonging to putative essential genes consisting of structural and DNA replication genes and separate clades belonging to nonessential genes categorized as a mix of hypothetical, miscellaneous, and tRNA genes (FIG. 3A). Clustering revealed that about two-thirds of Bas63 genes are nonessential (FIG. 3A). While this might indicate Bas63 is burdened with “deadweight” genes, the observation of conditional essentiality within nonessential genes—such as hypothetical genes gp114 and gp116—suggests that these genes may constitute a “toolbox” for thriving in varied environments.

A prevailing theme in phage genomes is genetic mosaicism, whereby loci accumulate from horizontal gene transfer events over time. It was investigated if mosaicism is evident from the gene essentiality profiles in Bas63 by overlaying the median fitness score for each gene over the genome map. Segments that are uniformly essential (e.g. positions 0 to ˜21,000) or nonessential (e.g. positions ˜46,000 to ˜66,000) were observed (FIG. 3B). Genes in the essential segments primarily code for structural proteins such as the tail- and capsid-related proteins. These genes mobilize as a cohesive unit, because their products often require interactions with other structural proteins. The nonessential segments contain genes encoding hypothetical proteins, lysis proteins such as holins, and enzymes such as ribonucleotide reductases and pyrophosphokinases. Genes for these proteins may function dependently with neighboring genes hence their co-localization. The diversity within these segments suggests that phages allocate highly flexible regions of the genome for acquiring additional functions that are separate from the essential regions to prevent disruption of core genes. Outside these segments, mosaicism was observed in regions containing interspersed small hypothetical genes with varying essentiality that may function more independently (e.g., positions ˜66,000 to ˜80,000) (FIG. 3B).

Binning Bas63 genes into 6 broad categories (DNA replication, structural, miscellaneous, hypothetical, lysis/membrane, and tRNA) revealed distinct gene essentiality within these categories. Across the 4 strains, DNA replication and structural genes were scored as essential (fitness<0) 83.3% and 73.1% of the time, respectively (FIG. 3C). Miscellaneous enzymes, with roles in cellular processes such as nucleotide and nicotinamide metabolism and redox reactions, and hypothetical proteins exhibited a mix of essentiality. Lysis/membrane proteins were mostly nonessential. Bas63 tRNAs were mostly nonessential (FIG. 3C). Because phage tRNAs are thought to compensate for host tRNA deficiencies or provide resistance to viral defense systems comprising of tRNA nucleases, these tRNAs are likely components within the conditionally essential toolbox that is leveraged in specific contexts.

In summary, PhageMaP can be applied to a non-model E. coli phage. Most Bas63 genes appear to be dispensable in our tested conditions, but these genes may serve beneficial functions when challenged to the appropriate condition.

### Example 4: PhageMaP Creates Gene Essentiality Profiles for a Large Phage with a Modified Genome

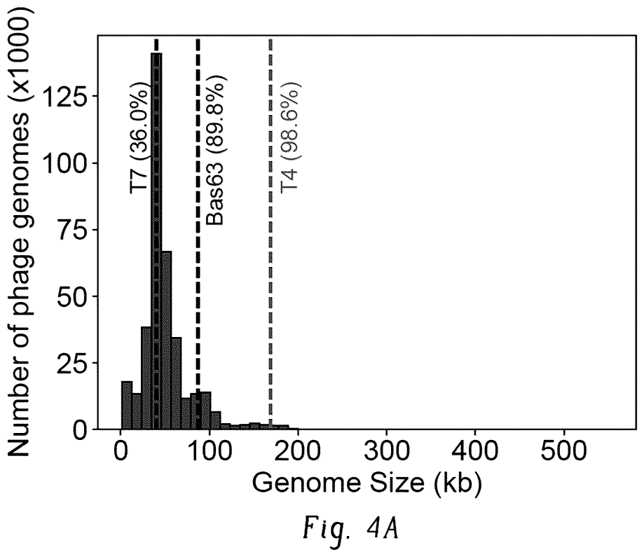

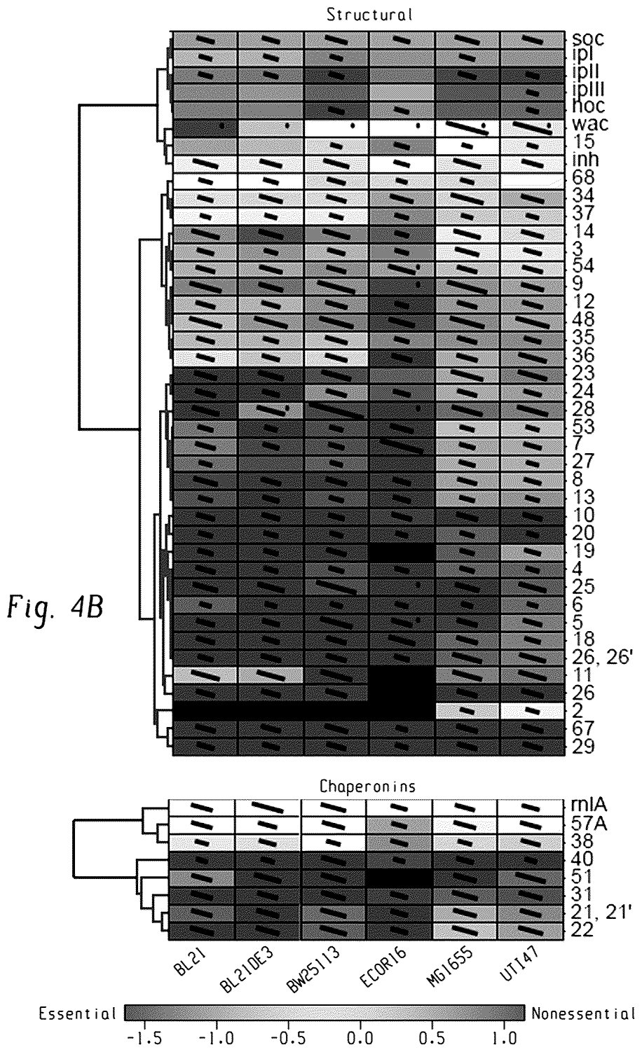

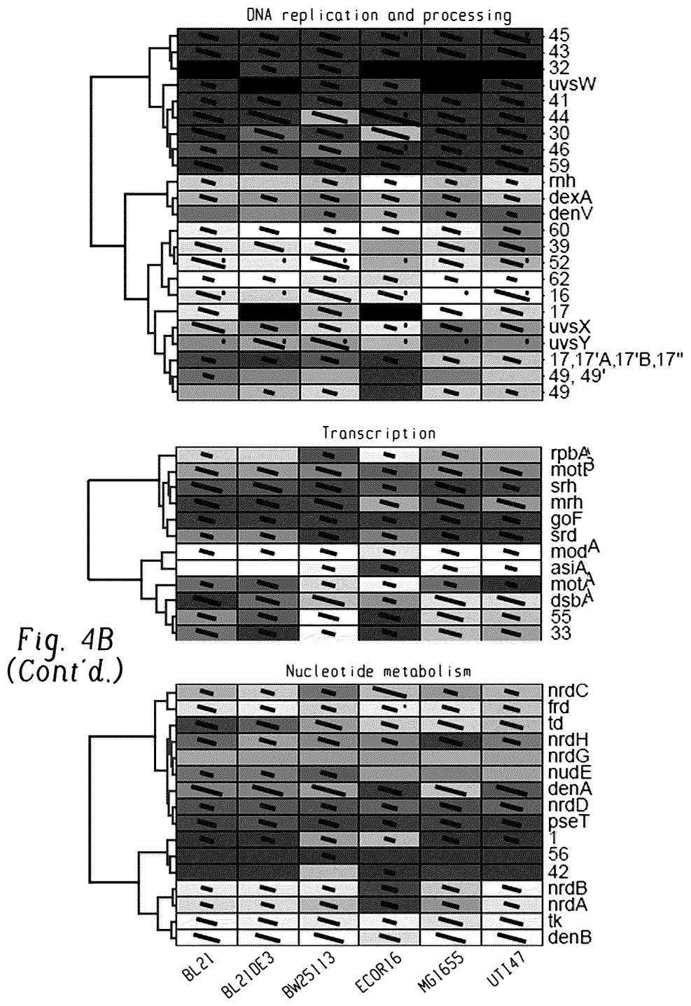

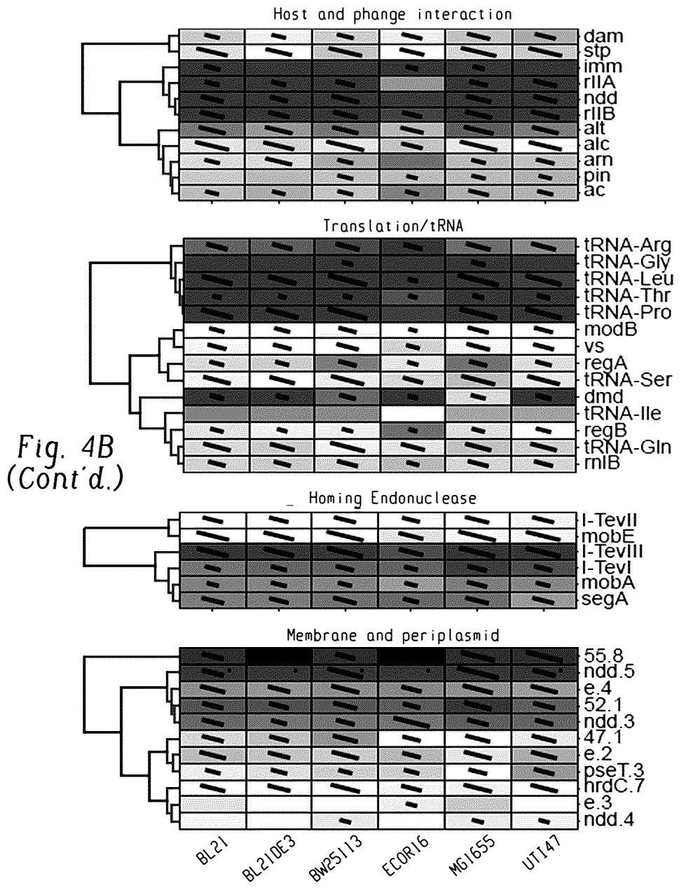

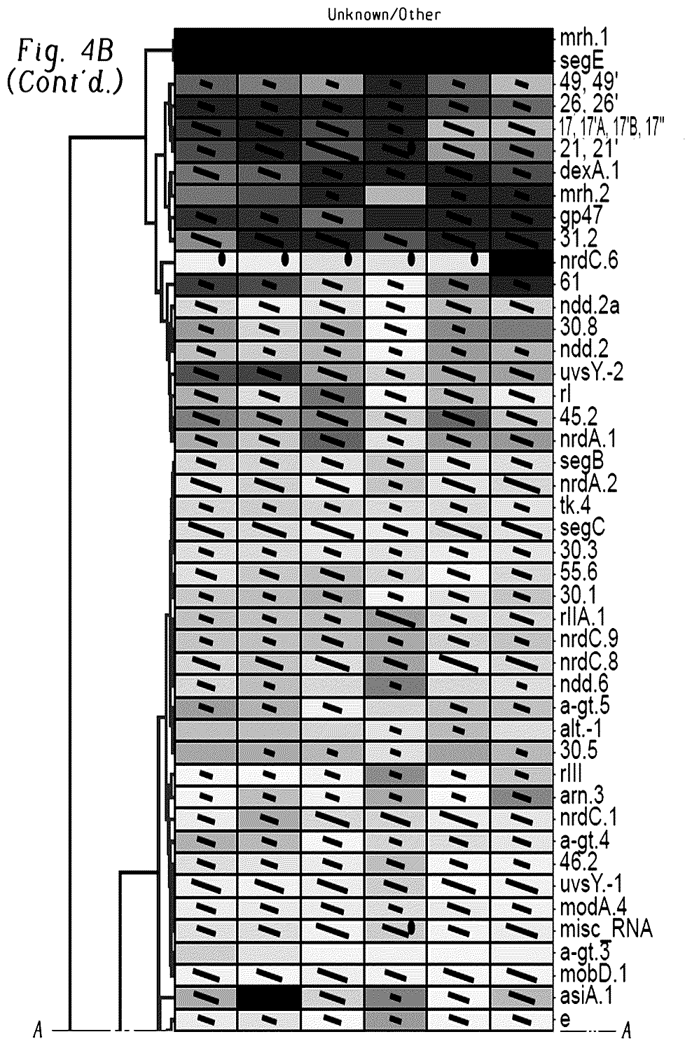

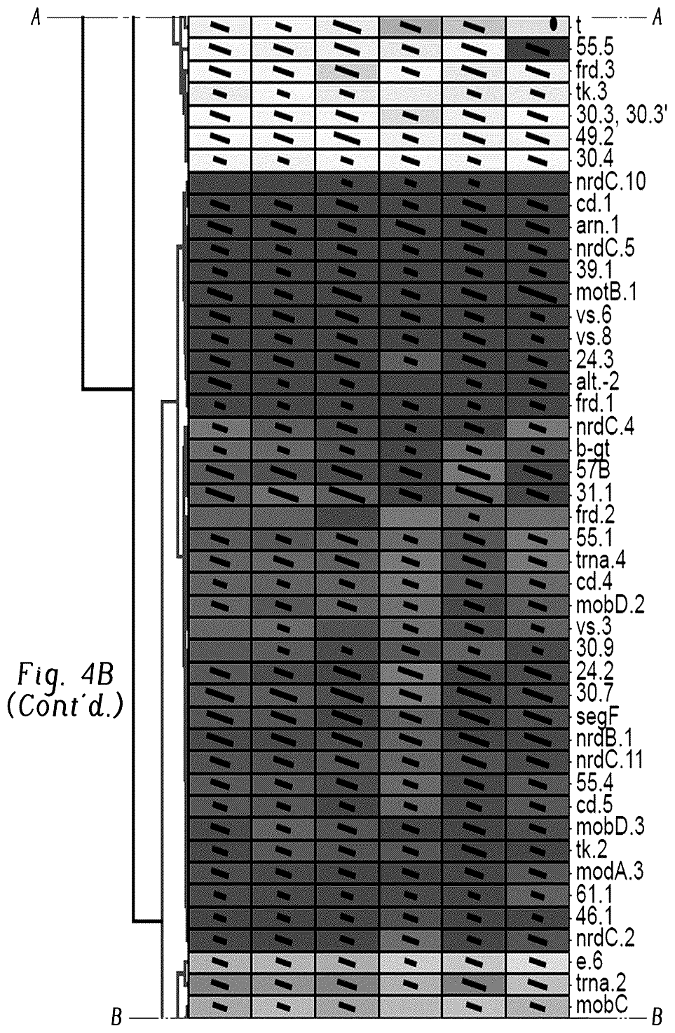

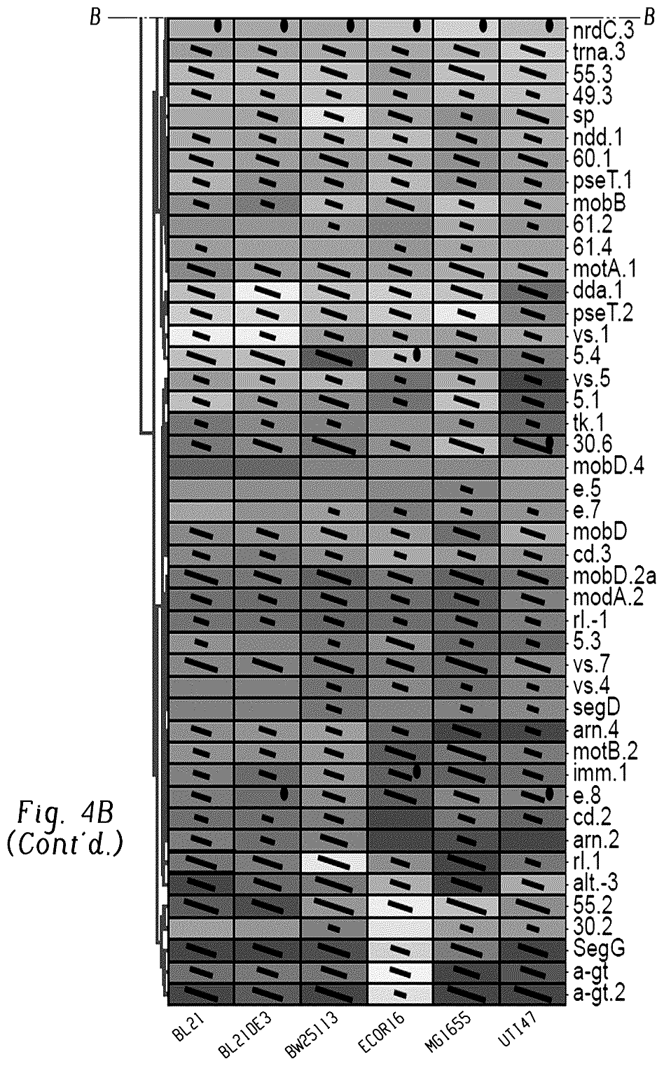

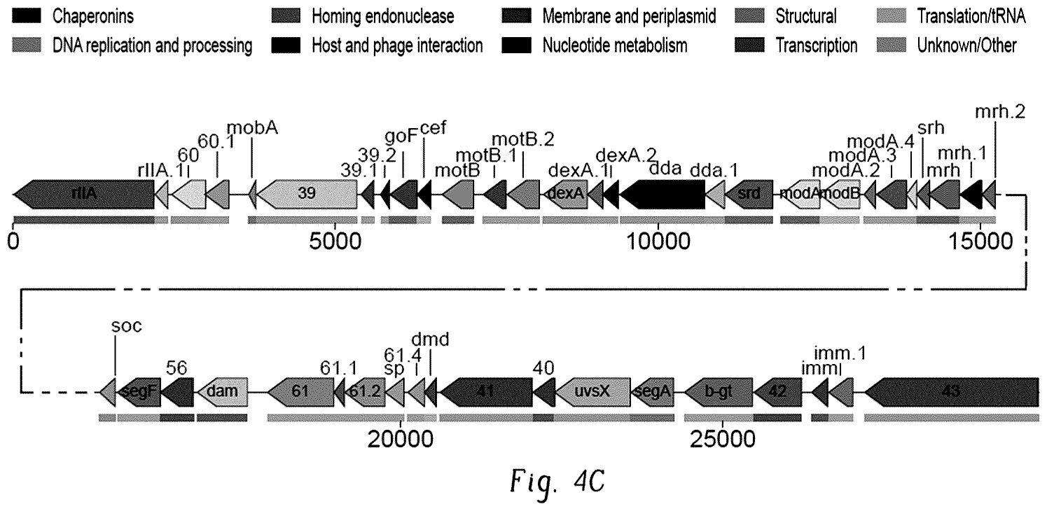

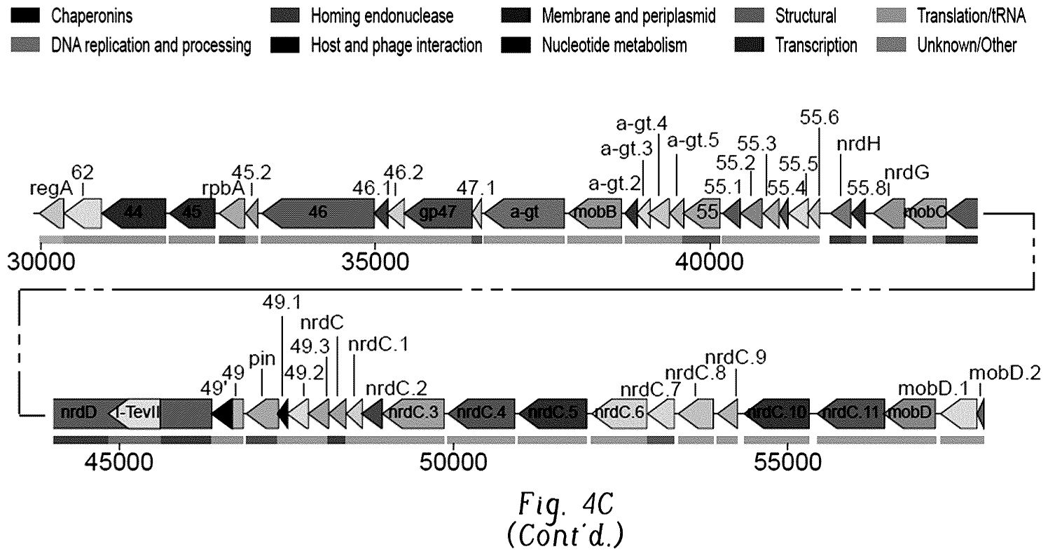

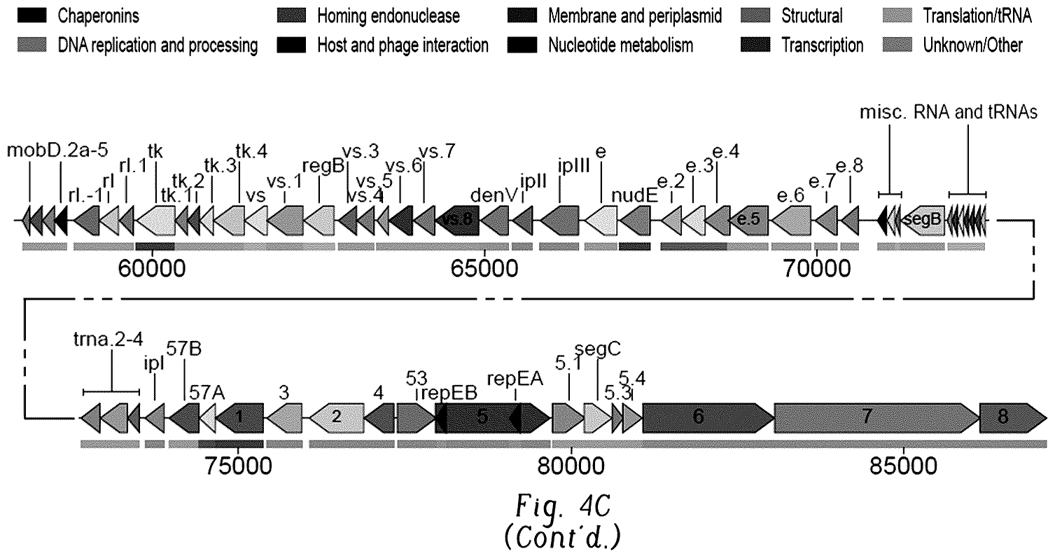

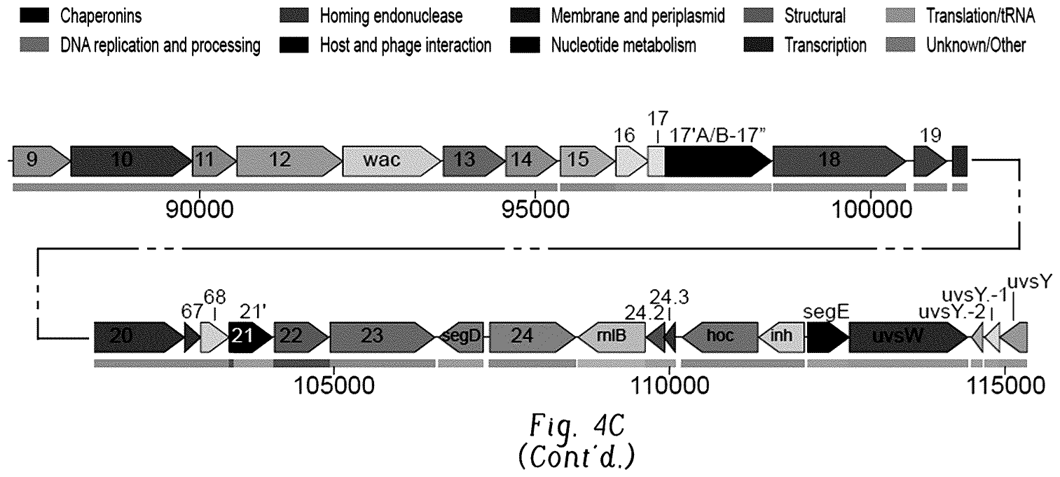

PhageMaP screening was next applied to T4 phage to further evaluate the genomic limits of the method. T4 was chosen for three reasons. First, with a 169 kb genome consisting of ˜300 genes, T4 exhibits genetic diversity comparable to some jumbo phages. Its genome length surpasses ˜98.6% of all complete or high-quality phage genomes (N=372,805) in the PhageScope database (FIG. 4A). Successful application to T4 would support the feasibility of PhageMaP for jumbo phages. Second, T4's genome is modified through cytosine 5-hydroxymethylation and glucosylation, which can inhibit Cas9 nuclease activity. This presents an opportunity to assess whether PhageMaP can generate phage libraries for modified genomes. Lastly, T4's extensive literature allows benchmarking of PhageMaP results against previous studies.

The T4 library was initially screened against ten E. coli strains in LB selection medium, but it was found that the results did not pass the reliability criterion (3Q=0.42; MED=0.22), prompting refinement of the experimental conditions. The T4 library was next screened against 6 E. coli strains in M9 minimal medium. These results passed the reliability criterion (3Q=−0.79; MED=0.48) and replicates generated reproducible scores (Pearson's r>0.92). To compare the consistency of results from both screens with prior knowledge, the genes were separated into three groups (Essential, Auxiliary/Nonessential, and Unknown) based on the putative essentialities outlined by Miller et al and analyzed fitness scores of genes within each group. Within the M9 condition, Essential genes generally had lower fitness scores (<0), Auxiliary/Nonessential genes are distinguished by their higher fitness scores (>0), and genes with previously Unknown essentiality appear to be primarily nonessential (fitness scores mostly >0). For the LB screen, while the Unknown genes were similarly identified as mostly nonessential, the distinction between Essential and Auxiliary/Nonessential genes was less clear. Thus, while the LB screen provided initial insights, repeating the assay in M9 minimal medium produced more reliable results. We proceeded to focus our analysis on the M9 dataset. Slower phage infections, alongside reduced likelihood of extracellular phage assembly in minimal media, may minimize essential genes appearing nonessential.

1606 fitness scores were obtained for 273 genes which resulted in 39% of the genes being categorized as essential (FIG. 4B). Using gene function designations described in the prior art, genes found within the Structural, Chaperonins, and DNA replication and processing categories are mostly essential while other categories such as Transcription and Nucleotide metabolism have a more diverse mix of essentialities (FIG. 4B). Within the Unknown/Other category, most genes appear to be nonessential (FIG. 4B). Some rare exceptions such as dexA.1, mrh.2, and ndd.2a appear to be essential genes but currently have unknown roles during phage infection. Like Bas63, gene essentiality had a highly mosaic pattern in most regiones except for those which include stretches of essential structural genes (FIG. 4C).

It was observed that dmd, an antitoxin to RnlA toxins60, is more essential in BW25113 and MG1655 than the other strains, likely due to their expression of RnlA (FIG. 4B, Translation/tRNA). Some genes with unannotated function (e.6, trna.2, and 55.5) are more essential in UTI47, presenting an opportunity to unveil the roles of previously uncharacterized genes (FIG. 4B, Unknown/Other). Conversely, asiA, a gene involved in regulation of host sigma 7061, is less essential in ECOR16, implying strain-level differences in transcription (FIG. 4B, Transcription). These findings reinforce the importance of experimenting on multiple hosts when describing gene essentiality.

Taken together, PhageMaP is applicable to large phages with modified genomes, paving the way for testing in less tractable, large phages with vast genetic diversity.

### Example 5: PhageMaP Identifies Anti-Phage Defense Counters and Triggers which can Inform Rational Phage Design

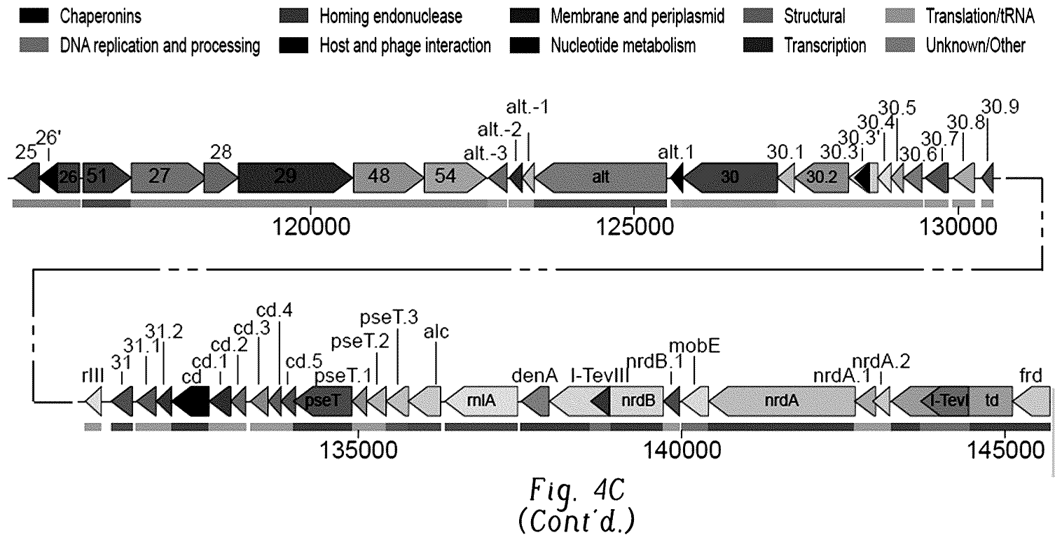

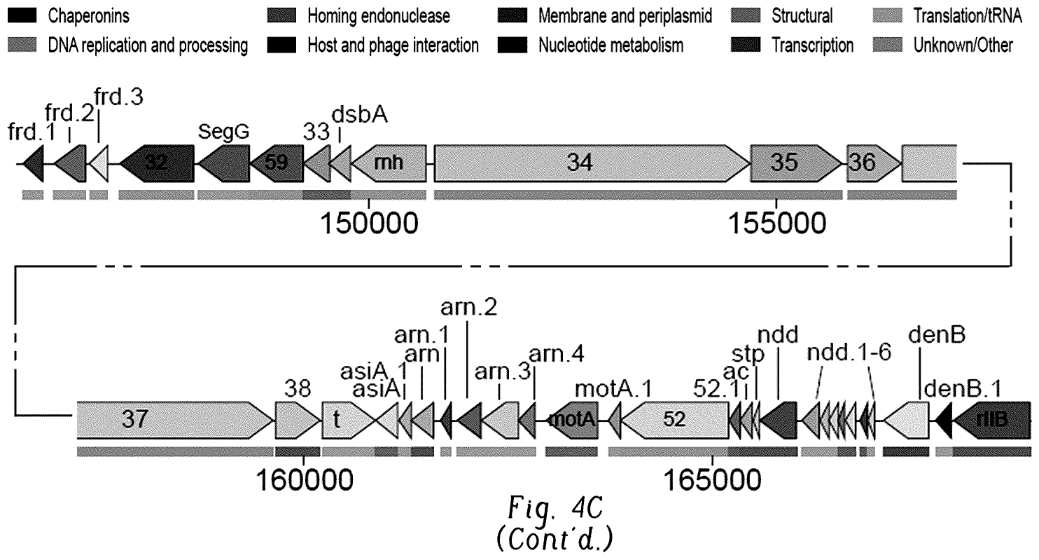

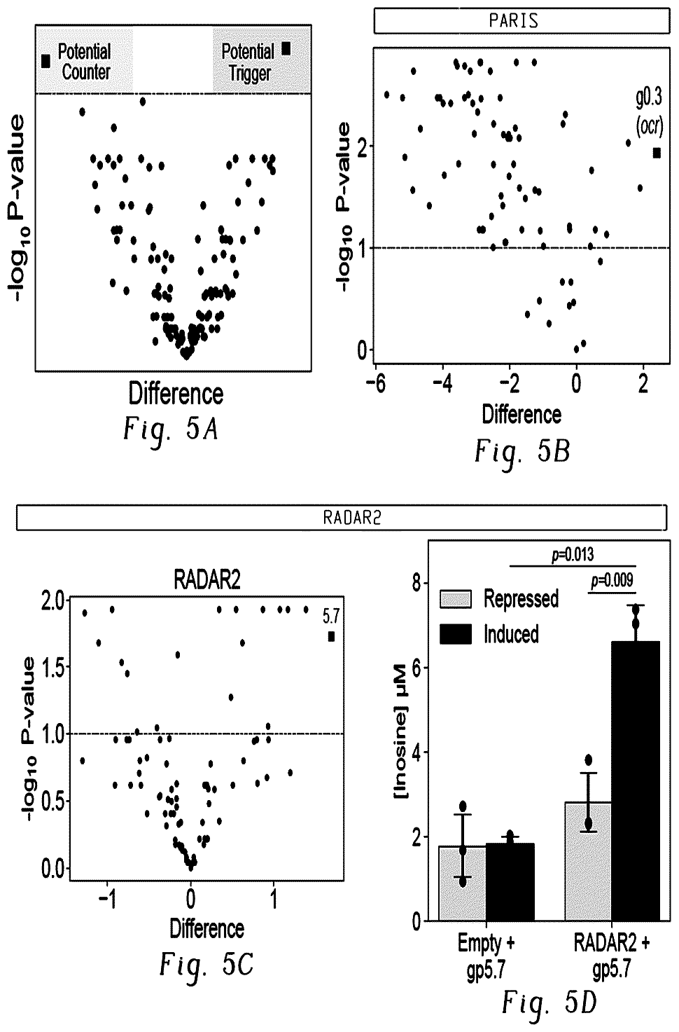

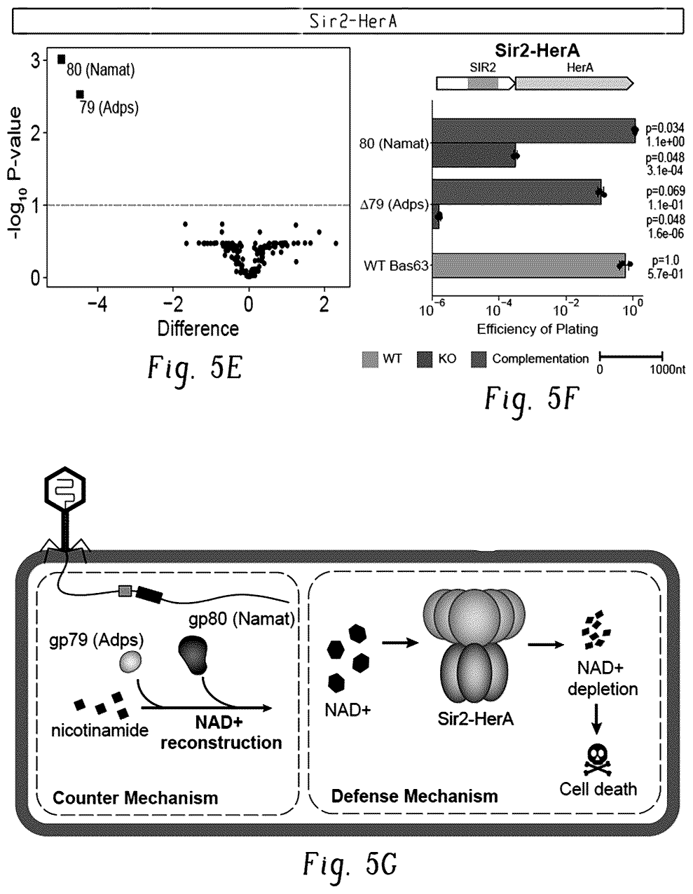

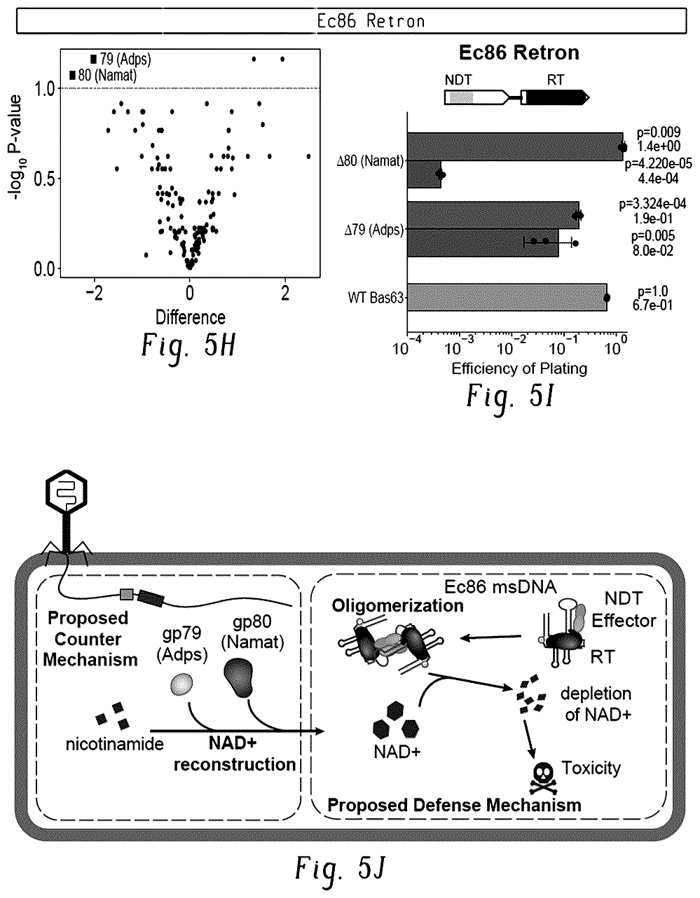

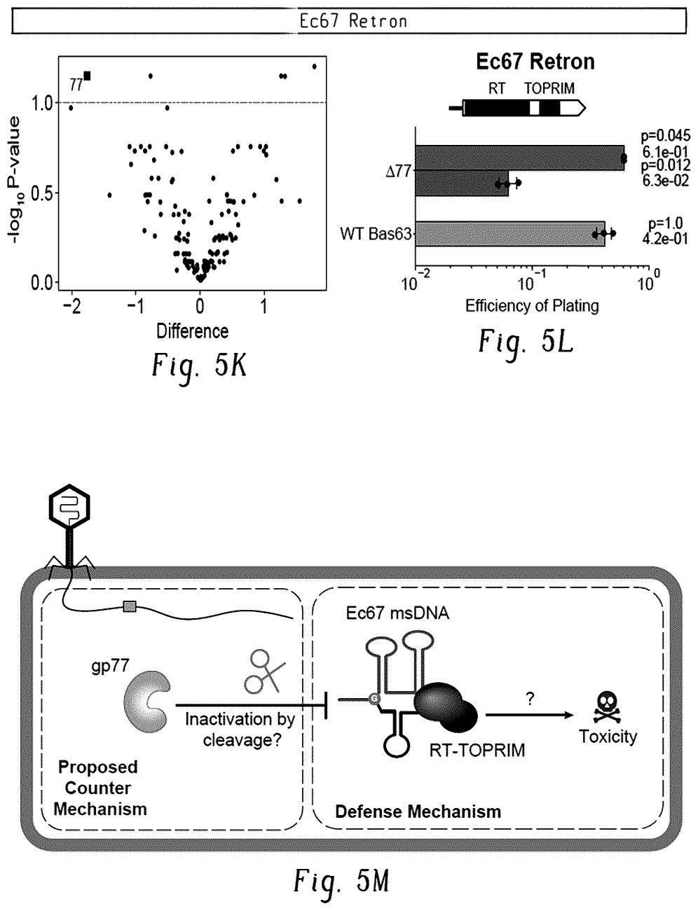

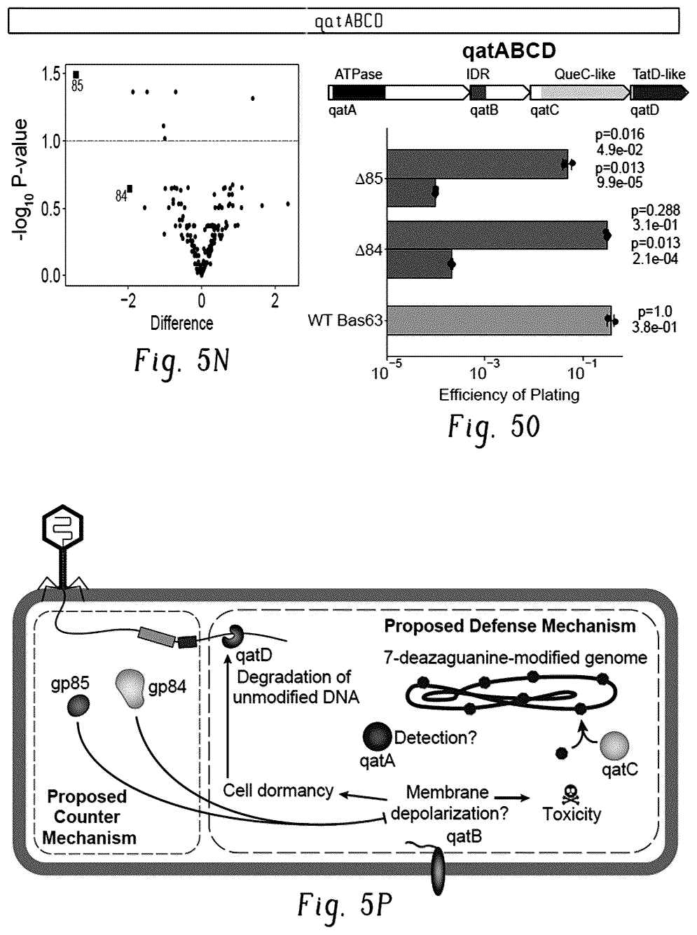

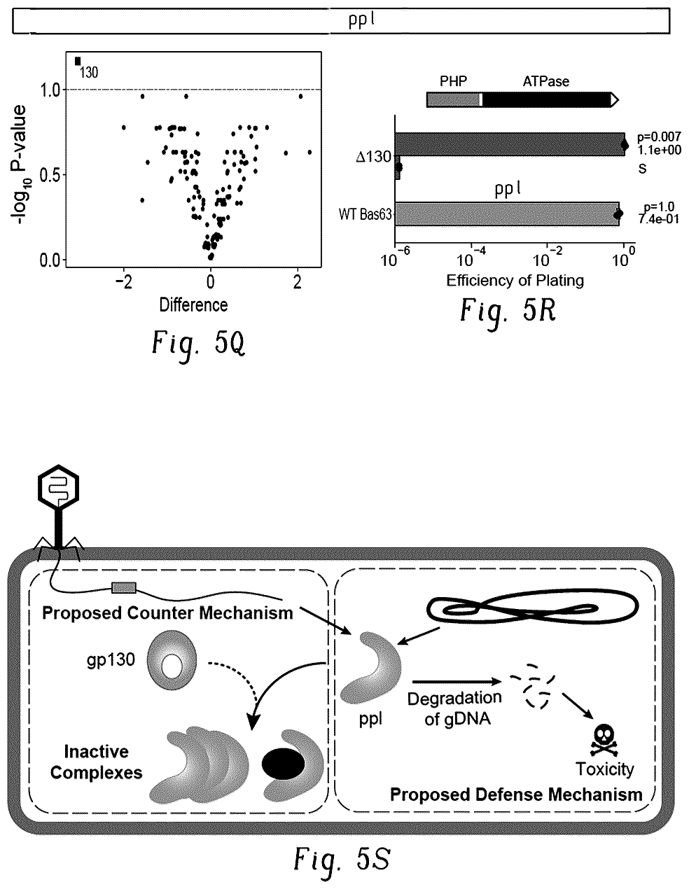

Recent years have seen an explosion of newly discovered and enzymatically diverse anti-phage defense systems. To counter bacterial immunity, phages evolved mechanisms to evade these defenses. PhageMaP enables systematic and efficient study of these interactions, many of which are poorly understood. Phage gene products can be “counters” or “triggers”. Counters inhibit host anti-phage defense to improve phage fitness and are more essential under anti-phage defense. Triggers activate host anti-phage defense to reduce phage fitness and are more nonessential under anti-phage defense (FIG. 5A).

The greater essentiality of ocr in strains containing intact Type I RM systems from the previous section demonstrates PhageMaP's ability to identify counters. To test if triggers can also be identified, selection experiments were performed using bacterial phage anti-restriction-induced system (PARIS) or Empty vector strains. PARIS is a toxin-antitoxin system that serves as a second line of anti-phage defense after RM systems are thwarted by phage counter-defense proteins such as ocr, inducing the release of the toxin. In PARIS strains, ocr knockouts should avoid triggering anti-phage activity and thus persist post-selection, leading to a higher fitness score compared to Empty vector. As expected, ocr barcodes were highly enriched (˜80% of reads) in the PARIS condition, validating PhageMap's ability to identify triggers (FIG. 5B).

To further explore phage-antviral interactions, T7 and Bas63 PhageMaP libraries were challenged against a panel of 33 anti-phage defense systems assembled as in the art. This panel is collectively represented in 32% of sequenced bacterial and archaeal genomes and provides a standardized background for identifying causal interactions by expressing each system in the same genetic context.

To validate counters, the plating efficiencies of wildtype phage and knockout variants were compared. Loss of effective counters would reduce the plating efficiency of the phage on the defense host relative to wildtype. Triggers are commonly validated through cell growth assays where expression of the trigger induces cellular toxicity. In practice, trigger validation can be challenging because reduced cell viability due to inherent phage gene toxicity or abortive infection, a common mechanism for anti-phage defense, is phenotypically ambiguous.

Gp5.7, an inhibitor of 6-dependent transcription in E. coli, is a predicted RADAR2 trigger (FIG. 5C). Since RADAR activity is characterized by the conversion of (d)ATP to (d)ITP, it was examined whether expression of gp5.7 in the presence of RADAR2 leads to inosine accumulation. A ˜2-fold increase in cellular inosine was observed upon gp5.7 induction in RADAR2, but not in Empty vector cells, confirming that gp5.7 activates RADAR2-mediated adenosine deamination (FIG. 5D). A prior study showed that gp5.7 also triggered nucleotide depletion from dCTP deamination and dGTP hydrolysis, suggesting an evolutionarily conserved response to transcriptional disruption.

For Sir2-HerA, g79 and g80 were identified as counters (FIG. 5E). Deletion of g79 or g80 led to a 10⁴-10⁶ reduction in plating efficiency in Sir2-HerA cells, which was restored when the genes were complemented (FIG. 5F). Sir2-HerA is a bipartite phage defense system that induces abortive infection through depletion of NAD+, a vital cofactor. In a recent study, genes 79 (Adps) and 80 (Namat) were both identified as enzymes involved in NAD+ reconstitution through a novel pathway. Specifically, Adps phosphorylates ADPr (ADP-ribose) to generate ADPr pyrophosphate (ADPr-PP). NAD+ is then synthesized by Namat through a reaction involving ADPr-PP and nicotinamide. NAD levels of Sir-HerA cells infected by wildtype Bas63 were reduced ˜18-fold relative to infected Empty vector cells. NAD depletion was even greater (˜31-fold) when cells were infected by Bas63 missing Adps and Namat. These results and those in literature demonstrate that Bas63 contains NAD+ synthesizing enzymes that regenerate NAD+ upon depletion by Sir2-HerA (FIG. 5G).

Adps and Namat were also identified as counters to the Ec86 retron and hhe defense systems with lower effectiveness than in Sir2-HerA (FIG. 5H). Ec86 retron plaquing efficiency was reduced ˜10-fold and ˜10³-fold when Adps and Namat were removed, respectively. These deficiencies are at least partially recovered upon complementation (FIG. 5D). Ec86 retron consists of a non-coding RNA (ncRNA), a nucleoside deoxyribosyltransferase-like (NDT) effector, and a reverse transcriptase (RT) that synthesizes a RNA-DNA hybrid molecule, multicopy single-stranded DNA (msDNA). These components assemble into oligomers to hydrolyze NAD+, inhibiting phage replication 82 (FIG. 5J). NAD levels of Ec86 retron cells infected by wildtype Bas63 were reduced ˜4-fold relative to infected Empty vector cells while infection by a double Adps and Namat knockout resulted in an 18-fold depletion. In the plating assays, Adps appeared to be less important for evasion of Ec86 retron defense (FIG. 5I). NAD depletion by Ec86 retron results in release of nicotinamide and covalent attachment of ADPr to the effector active site. Without being held to theory, it is speculated that ADPr is pyrophosphorylated upon release from this catalytic site, obviating the need for pyrophosphorylation by Adps. On the other hand, Sir2-HerA releases un-pyrophosphorylated ADPr and therefore requires Adps.

For hhe, consisting of a single gene containing helicase and nuclease domains, deletion of either counter yielded a reduction in plaque size. Pooled competition assays were performed with WT Bas63 and Bas63Ag79 and it was found that Bas63Ag79 is less fit than wildtype Bas63 in hhe cells, but this effect was negated after complementation, suggesting that the hhe defense system may also involve NAD+ depletion. However, no change in NAD levels was observed in hhe cells after infection with either wildtype Bas63 or a double Adps-Namat knockout, indicating that the mechanism of hhe defense is not NAD+ depletion and that Adps and Namat's roles in countering hhe defense are distinct from NAD reconstitution. Intriguingly, infection of hhe cells with Adps or Namat knockout phages resulted in reduced plasmid concentration. This reduction was not observed when cells were infected with wildtype Bas63 or when cells contained the Empty vector plasmid, suggesting a DNA degradation mechanism that is mitigated by Adps and Namat. Further studies are necessary to elucidate the specific roles of Adps and Namat in hhe defense. Taken together, these data highlight the convergence of phage counterstrategies to distinct anti-phage defense systems and the ability of PhageMaP to identify co-essential genes that function within a shared pathway.

For Ec67 retron, gp77 was identified as a counter (FIG. 5K). Deletion of gp77 resulted in a 10-fold reduction in plating efficiency, which was restored upon complementation (FIG. 5L). Ec67 retron consists of a reverse transcriptase-endonuclease fusion gene and a ncRNA that produces msDNA. Gp77 contains a GIY-YIG catalytic domain which is involved in DNA repair, transfer of retroelements, or degradation of foreign DNA. It has been shown that degradation of the msDNA precursor is a mechanism for retron inactivation, thus gp77 may cleave and inactivate the Ec67 msDNA (FIG. 5M). In support of this, Ec67 msDNA (or more accurately, ssDNA of the msDNA) levels are reduced by ˜4-fold upon induction of gp77.

For gatABCD, PhageMaP identified g85 and g84 to be counters (FIG. 5N). Deletion of either gene resulted in a 10⁴-10⁵ reduction of plating efficiency that is largely restored after gene complementation (FIG. 5O). QatABCD comprises of four genes: QatA, which contains an ATPase domain; QatB, an uncharacterized protein with structural similarity to transmembrane proteins; QatC, which contains a QueC domain possibly involved in DNA modification; and QatD, a TatD nuclease which has a role in DNA fragmentation. The exact mechanism for gatABCD defense is unknown. Genes 85 and 84 encode membrane integrity protector proteins that share high sequence homology (>80%) to RIIA/B found in T4. RIIA and RIIB inhibit RexAB-mediated membrane depolarization to allow T4 infection of lambda lysogens. Without being held to theory, it is proposed that gatABCD inhibits Bas63 replication through a similar mechanism where membrane depolarization by QatB induces cell death or dormancy and activates QatD degradation of unmodified phage genomes (FIG. 5P). The host genome is protected due to DNA modification by QueC (FIG. 5P). QatA may be responsible for regulation of the anti-phage defense process, as is common with ATPases in other systems. Further studies are necessary to decipher the exact roles gp84 and gp85 have in gatABCD defense.

PhageMaP identified g130 as a ppl counter (FIG. 5Q). Deletion of gene 130 severely reduced Bas63 plaque size and plating efficiency, but this phenotype was reversed upon gene complementation (FIG. 5R). Ppl is an uncharacterized anti-phage system consisting of a single gene containing an ATPase domain and a Polymerase and Histidinol Phosphatase (PHP) domain with putative phosphodiester-hydrolyzing function. Gp130 shares high structural similarity to Ro60, a regulator of RNA processing and decay through enzymes such as exoribonucleases. To better understand how gp130 may affect ppl activity, we performed formaldehyde cross-linking analysis in cells expressing gp130 and ppl together, as well as with each protein individually. Formaldehyde cross-linking stabilizes protein complexes in situ and enables detection of interactions involving gp130 and ppl. It was found that ppl assembles into a dominant, high-molecular weight (>250 kDa) species in the presence of gp130 but not with the sfGFP control. These complexes possibly contain inactivated ppl. Since a similarly sized complex is not evident for gp130, gp130 may be acting through an indirect mechanism. Without being held to theory, we speculate that ppl degrades phage and host DNA upon infection, but this process can be inhibited by gp130, which indirectly induces formation of inactive ppl complexes (FIG. 5S).

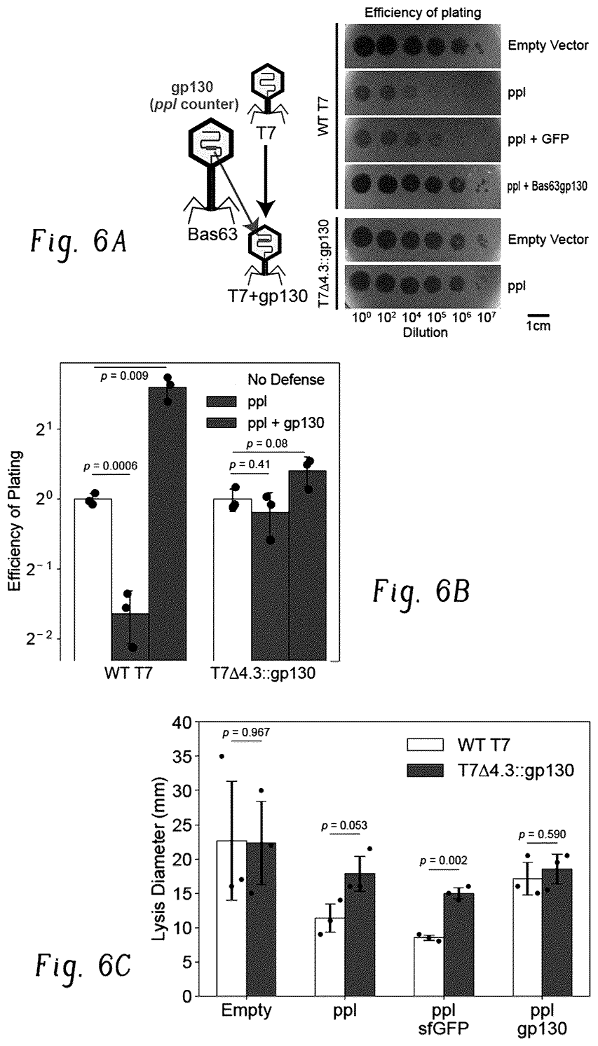

To test if counters identified from one phage can improve infectivity of a separate ineffective phage, we sought out a defense strain with a PhageMaP-identified counter that is fully susceptible to only either Bas63 or T7. Gp130 provides Bas63 with ppl immunity, a strain which has resistance to T7 (FIG. 5R). ppl impaired plaque formation for T7 compared to the No-defense control (FIG. 6A-C). It was tested if this phenotype is reversed by expression of gp130 from a plasmid or directly from the phage genome by replacing the nonessential g4.3 with gp130. Expression of gp130 but not a GFP control from a plasmid restored the T7 plaquing phenotype to that of the No-defense control (FIG. 6A). Interestingly, expression of gp130 from a plasmid improved plaquing efficiencies past the efficiencies seen in the No-defense control, suggesting gp130 may have a general role in robust phage growth (FIG. 6B). Expression of gp130 directly from the T7 genome improved plaquing efficiency ˜3-fold on the ppl strain compared to wildtype (FIG. 6A-B). Wildtype T7 forms small, turbid plaques on the ppl strain in the absence of gp130 (FIGS. 6A and C). Expression of gp130 either from a plasmid or the genome resulted in larger (57-76% diameter increase) and clearer lysis phenotypes (FIGS. 6A and C). These data indicate that counters are transferable and demonstrate how PhageMaP can be leveraged to engineer more efficacious phages.

### Discussion

High-throughput genetic screens have revolutionized the way we study phage-host interactions. PhageMaP is a scalable, high-throughput, and systematic method to generate and screen barcoded phage knockout libraries. Due to pooled format and the use of pre-mapped barcodes found within the phage genomes as a quantitative readout, PhageMaP is suitable for simultaneous interrogation of the conditional essentiality of all phage genetic elements—coding and noncoding—in many experimental conditions all in one sequencing experiment. Because the phage library only needs to be created once in a permissible strain and testing is host-agnostic, conditional essentiality testing on recalcitrant hosts is simplified compared to methods that require genetic manipulation of the target strain prior to testing or utilize host abundances as readout. The use of Cas9-RecA-mediated HR to efficiently introduce mutations obviates individually synthesizing phage knockouts through methods such as Gibson Assembly or Golden Gate Assembly which are generally tedious, high in reagent consumption, lower throughput, and not scalable to larger phage genomes that are of particular interest in gene essentiality studies. PhageMaP is a discovery tool for identification of novel phage-host molecular interactions and functional characterization of genes with previously unknown function.

For proof of concept, the conditional essentiality of coliphage T7 was examined. With decades of research performed using this model phage, we reasoned it is a good option to benchmark the effectiveness of PhageMaP. Indeed, gene essentiality patterns that corroborate the years of findings can be determined in a single sequencing experiment despite using a highly biased library. Due to the planktonic nature of the screens, there is the possibility of acquiring artefacts due to in-solution gene complementation as observed with the T7 tail fiber gene. After testing on diverse phage defense hosts, weak counters and triggers were identified for 3 defense systems which were previously unknown. Two of these genes (4.5 and 0.3) have previously annotated function. Here the data describes possible alternative modes of antiphage defense evasion by those genes. Because systems such as RADAR1/2 originate from separate genera, it is unlikely that T7 evolved counters to the exact variant of the defense system tested, explaining the small effect sizes. However, the ability to capture these weak interactions highlight the sensitivity of PhageMaP. More robust inhibitors can be generated using metagenomic mining and directed evolution approaches.

To demonstrate generalizability and the ability to scale to large phages, we applied PhageMaP to Bas63 and T4 and performed similar testing as T7. Notably, T4 also consists of a modified genome. Screening on common laboratory strains resulted in a comprehensive gene essentiality map for most genes in Bas63 and T4. Expected trends for highly essential structural and DNA replication genes were observed. Counters and triggers for 7 different phage defense systems were identified and validated. The ability of PhageMaP to capture larger effect sizes was confirmed and it was found that the fitness scores may have a rough correlation to results obtained from secondary forms of analyses such as plaque assays. The results from a recent study identifying Bas63 genes 79 and 80 as counters for Sir2-HerA-mediated NAD+ depletion was corroborated. Novel molecular interactions between discovered counters and defense systems gatABCD, Ec67 retron, Ec86 retron, hhe, and ppl are proposed. Like Sir2-HerA, Ec86 retron and hhe defenses are also inhibited by gp79 and gp80, demonstrating the ability of the same set of genes to counter different mediators of immune response.

The PhageMaP datasets have exciting applications in rational phage engineering. The identification of broadly nonessential genes facilitates construction of a minimal phage genome which can serve as a scaffold for introduction of non-native genes that boost phage efficacy in a certain application. T his purpose is complemented by the discovery of counters and activators of anti-phage systems through the high-throughput conditional essentiality testing. Such counters can be added, and triggers can be removed from the phage genome if beneficial for activity on a target host.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of preparing a barcoded target phage variant library, comprising
providing a donor plasmid library, wherein each member of the donor plasmid library is a bacterial vector comprising a single guide RNA (gRNA) spacer sequence, a 3′ homology arm sequence, and 5′ homology arm sequence, wherein the gRNA spacer and homology arms define a Cas nuclease cleavage site at a genomic locus in the target phage,
inserting a barcode insert into each member of the donor plasmid library between the 3′ and 5′ homology arms to provide a barcoded donor plasmid library, wherein the barcode insert comprises a barcode flanked by barcode primer binding sites, and
transforming a host cell with the barcoded donor plasmid library to provide a transformed host cell, wherein the host cell is engineered to express a Cas nuclease and a recombinase,
infecting the transformed host cell with a population of target phage to provide the barcoded target phage variant library, wherein double stranded cleavage of the target phage genomes by the Cas nuclease-gRNA and subsequent recombinase-mediated homologous recombination with the donor plasmid inserts the barcodes into the genomic loci in the target phage, wherein insertion of the barcodes at the genomic loci disrupts the function of the genomic loci, and
isolating the barcoded target phage variant library.

2. The method of claim 1, wherein the host cell does not express an endogenous recombinase.

3. The method of claim 1, wherein prior to infecting, the host cell is transformed with a plasmid for expression of the Cas nuclease and the recombinase.

4. The method of claim 1, wherein the barcoded target phage variant library comprises members with barcode insertions in protein coding and/or non-coding genomic loci.

5. The method of claim 1, wherein the barcoded target phage variant library comprises members with barcode insertions in at least 50% of target phage genes.

6. The method of claim 1, further comprising mapping the barcodes of the members of the barcoded target phage variant library to their loci in the target phage genome by sequencing the barcoded plasmid library.

7. The method of claim 6, wherein each target gene locus is associated with 10 to 100 barcodes.

8. The method of claim 1, wherein at least a portion of the target gene loci are in intergenic regions.

9. The method of claim 1, wherein the target phage is a phage with a sequenced genome.

10. The method of claim 1, wherein the target phage is a lysogenic phage.

11. A method of multiplexed, targeted phage genome manipulation and profiling, comprising providing a barcoded target phage variant library, wherein each member of the barcoded target phage variant library comprises a barcode inserted at a genomic locus in the target phage, wherein insertion of the barcode at the locus disrupts the function of the locus,
challenging a panel of susceptible bacterial hosts or a single susceptible bacterial host under a plurality of conditions with the barcoded target phage variant library, and
quantifying the pre-challenge and post-challenge abundances of one or more phage variants from the barcoded target phage variant library.

12. The method of claim 11, further comprising preparing a map of genomic locus essentiality by assigning a fitness score to each variant phage based on the pre-challenge and post-challenge abundances, wherein the fitness score is a measure of genomic locus essentiality.

13. The method of claim 12, wherein the genomic loci in the variant phage are classified as essential, non-essential or conditionally essential.

14. The method of claim 13, wherein the essential and nonessential target genomic loci have fitness scores positioned on opposing ends of a numerical fitness scale.

15. The method of claim 13, wherein each conditionally essential target genomic locus has at least one fitness score that is positioned on an opposing end of a numerical fitness scale from the other fitness score(s) involving the same or related genomic locus.
